(19) United States
(12) Patent Application Publication
(10) Pub. No.: US 2006/0148735 A1
(43) Pub. Date: Jul. 6, 2006

(54) Methods for treating, preventing, or reducing cardiac disorders using fadd inhibitors

(75) Inventors: Anthony Rosenzweig, Newton, MA (US); Wei Chao, Andover, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 10/534,314

(22) PCT Filed: Nov. 13, 2003
(86) PCT No.: PCT/US03/36624

§ 371 Date: Oct. 24, 2005

(30) Foreign Application Priority Data
Nov. 15, 2002 (US) 60426755

(51) Int. Cl. A61K 48/00

(52) U.S. Cl. 514/44

(57) ABSTRACT

The present invention relates to methods for treating or preventing cardiac disorders such as heart failure, by administering to a mammal a therapeutically effective amount of an FADD inhibitor. According to the present invention, the FADD inhibitor is anti-apoptotic, anti-inflammatory, or both. Also disclosed are screening methods that make use of FADD for the identification of novel therapeutics for treating or preventing cardiac disorders.

### FIELD OF THE INVENTION

The field of this invention is the treatment, prevention, or reduction of cardiac disorders. In particular, the invention relates to methods for preventing or reducing cardiomyocyte apoptosis, cardiac inflammation, and ischemic reperfusion injury associated with cardiac disorders, such as myocardial infarction, heart failure, and organ transplantation. Further, methods are provided for identifying candidate compounds useful for treating, preventing, or reducing cardiac disorders.

### BACKGROUND OF THE INVENTION

Myocardial infarction (MI) is a major cause of morbidity and mortality in Western cultures. Although this condition is typically treated by the restoration of blood flow (reperfusion) to the affected area, the process of reperfusion itself exacerbates the damage to cardiomyocyte cells (CM) in a process known as ischemia-reperfusion injury (IRI). Minimizing this damage could therefore maximize the benefits of reperfusion therapy following acute infarction.

In animal models, cardiac IRI is characterized by a significant proportion of the damaged area being affected by inflammation and apoptosis. Recent experimental data have further suggested a causative role for both of these processes in cardiac injuries associated with a number of additional pathological disorders, including, for example, injuries that result from chronic and acute ischemia, myocarditis, congestive heart failure, cardiac transplantation, autoimmune disorders, as well as a variety of cardiac surgical procedures such as cardiopulmonary bypass surgery. Thus, methods for reducing apoptosis of cardiomyocytes and cardiac inflammation are needed.

### SUMMARY OF THE INVENTION

In general, the present invention features methods and compositions for the treatment, prevention, or reduction of cardiac disorders.

In a first aspect, the invention features a method of reducing or preventing apoptosis of cardiomyocytes (e.g., adult cardiomyocytes), by administering to such cardiomyocytes an effective amount of an anti-apoptotic FADD inhibitor. The invention also features a method for treating, reducing, or preventing cardiac inflammation in a mammal by administering an effective amount of an FADD protein or FADD inhibitor to the mammal. In all aspects of the present invention, the FADD inhibitor may be anti-apoptotic, anti-inflammatory, or both. The FADD inhibitor may be, for example, a dominant negative FADD protein, which is both anti-apoptotic and anti-inflammatory. Preferably, the mammal being treated is a human.

The invention also provides a method for treating, reducing, or preventing ischemic reperfusion injury to the heart of a mammal by administering to the mammal an effective amount of an FADD protein, an anti-apoptotic FADD inhibitor, or an anti-inflammatory FADD inhibitor. The ischemic reperfusion injury treated or prevented by this method may be acute; for example, the ischemic reperfusion injury may result from a myocardial infarct. Alternatively, it may be iatrogenically-induced; for example, the ischemic reperfusion injury may result from cardiac surgery, coronary artery bypass surgery, valve replacement surgery, or percutaneous transluminal coronary intervention, including angioplasty or stenting. The iatrogenically-induced ischemic reperfusion injury may also result from heart transplantation.

In yet another aspect, the invention provides a method for treating, reducing, or preventing a cardiac disorder in a mammal by administering to the mammal an effective amount of an FADD protein, an anti-inflammatory FADD inhibitor, or an anti-apoptotic FADD inhibitor. The cardiac disorder may result from a chronic ischemia injury, an acute ischemia injury, an ischemia-reperfusion injury, a myocardial infarction, myocarditis, heart failure, cardiac transplantation, or an autoimmune disorder. This method is particularly useful to treat, reduce, or prevent heart failure.

In another aspect, the invention features a method for preparing a donor cardiomyocyte for transplantation into a recipient, by contacting the cardiomyocyte with an anti-apoptotic FADD inhibitor (e.g., a nucleic acid encoding a dominant negative FADD protein), an anti-inflammatory FADD inhibitor, or an FADD protein. The donor cardiomyocyte may be a single cell, or alternatively, may be in a cardiac tissue or a heart to be transplanted into a recipient. Accordingly, the invention also features a cardiomyocyte, a cardiac tissue, or a heart expressing an anti-apoptotic FADD inhibitor, an anti-inflammatory FADD inhibitor, or an FADD protein. For example, a cardiomyocyte, cardiac tissue, or heart of the invention may express a dominant negative FADD protein. Preferably, the cardiomyocyte, the cardiac tissue, or the heart is from a human or a pig.

In yet another aspect, the invention provides a method for identifying a candidate compound for reducing or preventing apoptosis of cardiomyocytes, or alternatively, for treating, reducing, or preventing cardiac inflammation, ischemic reperfusion injury, or a cardiac disorder, such as heart failure. The method involves the steps of: (a) contacting an FADD expressing cardiomyocyte with a candidate compound; and (b) measuring FAD [symbol] gene expression or FADD protein activity. A candidate compound that reduces the expression or activity of FADD, relative to a cardiomyocyte not contacted with the candidate compound, is identified as useful for reducing or preventing apoptosis of cardiomyocytes, or alternatively, for treating, reducing, or preventing cardiac inflammation, ischemic reperfusion injury, or a cardiac disorder.

In preferred embodiments, the FAD [symbol] gene is an FAD [symbol] fusion gene. In other embodiments, step (b) involves the measurement of FAD [symbol] mRNA or protein. Preferably, the cardiomyocyte is a mammalian cell (e.g., rodent cell).

In a related aspect, the invention provides another method for identifying a candidate compound for reducing or preventing apoptosis of cardiomyocytes, or alternatively, for treating, reducing, or preventing cardiac inflammation, ischemic reperfusion injury, or a cardiac disorder, such as heart failure. This method involves the steps of: (a) contacting an FADD protein with a candidate compound; and (b) determining whether the candidate compound binds the FADD protein and inhibits FADD activity. Candidate compounds that bind and inhibit FADD activity are identified as useful for reducing or preventing apoptosis of cardiomyocytes, or alternatively, for treating, reducing, or preventing cardiac inflammation, ischemic reperfusion injury, or a cardiac disorder.

In preferred embodiments, the method also tests the ability of the candidate compound to reduce expression of the FADD gene in a cell, for example, a mammalian cell such as a rodent or human cell. Most preferably, the FADD is human FADD.

In another aspect, the invention also provides a kit containing (a) a vector expressing a nucleic acid encoding an anti-apoptotic FADD inhibitor and (b) instructions for delivery of the vector to a cardiomyocyte, cardiac tissue, or heart under conditions suitable for reducing or preventing apoptosis of cardiomyocytes. The invention also provides a kit containing (a) a vector expressing a nucleic acid encoding an anti-inflammatory FADD inhibitor or an FADD protein; and (b) instructions for delivery of the vector to a cardiomyocyte, cardiac tissue, or heart under conditions suitable for treating, reducing, or preventing cardiac inflammation. The invention further provides a kit containing (a) a vector expressing a nucleic acid encoding an anti-apoptotic FADD inhibitor, an anti-inflammatory FADD inhibitor, or an FADD protein; and (b) instructions for delivery of the vector to a cardiomyocyte, cardiac tissue, or heart under conditions suitable for treating, reducing, or preventing ischemic reperfusion injury or a cardiac disorder (e.g., heart failure). Optionally, the cardiomyocyte, the cardiac tissue, or the heart may be donor material to be transplanted into a recipient and may be cultured and maintained in vitro. Alternatively, the cardiomyocyte, the cardiac tissue, or the heart is in a mammal, preferably a human that has a cardiac disorder.

As used herein, by “FADD” is meant any polypeptide having at least 50%, 70%, 80%, 90%, 95%, or even 99% sequence identity to 10, 20, 35, 50, 100, 150, or more than 150 amino acids of the wild type human FADD protein and having biological activity (e.g., anti-inflammatory).

By “anti-apoptotic FADD inhibitor” is meant any compound that prevents FADD-mediated apoptosis or reduces the levels of FADD-mediated apoptosis when administered to a cell (e.g., cardiomyocyte), as compared with an equivalent untreated control. Such reduction or degree of prevention is at least 5%, 10%, 20%, 40%, 50%, 60%, 80%, 90%, 95%, or 100% relative to the untreated control, as measured by any standard technique. Preferably the anti-apoptotic FADD inhibitor also has anti-inflammatory properties.

By “anti-inflammatory FADD inhibitor” is meant any compound that treats, reduces, or prevents FADD-mediated cardiac inflammation when administered to a mammal relative to an equivalent untreated control; such treatment, reduction or degree of prevention is at least 5%, 10%, 20%, 40%, 50%, 60%, 80%, 90%, 95%, or 100% compared to the untreated control, as measured by any standard technique. The anti-inflammatory FADD inhibitor may or may not be an anti-apoptotic FADD inhibitor.

By “cardiac disorder” is meant any pathological condition resulting in an injury to the heart, or cardiac tissue. Such injuries may result from chronic or acute ischemic injuries, ischemia-reperfusion injuries, myocardial infarcts, myocarditis, heart failure, surgeries such as cardiac transplantation, autoimmune disorders, or infectious disorders.

By “reduces expression of a FADD gene or activity of a FADD protein” is meant to decrease expression or activity of FADD relative to control conditions. This reduction may be, for example, a decrease of least 2-fold, 3fold, 5-fold, 10-fold, 100-fold, or even 1000-fold or greater, relative to control conditions.

By “reducing or preventing apoptosis” is meant preventing apoptosis or reducing the levels of apoptosis in a cardiomocyte as compared with an equivalent untreated control; such reduction or degree of prevention is at least 5%, 10%, 20%, 40%, 50%, 60%, 80%, 90%, 95%, or 100% as measured by any standard technique. Standard techniques include for example DNA laddering, TUNEL assay, flow cytometry for DNA content, cell death Elisa, caspase activity, or detection of surrogate markers of apoptosis by immunohistochemistry, Western or Northern analysis.

By “treating, reducing, or preventing cardiac inflammation” is meant preventing inflammation or decreasing the level of inflammation in a heart or cardiac tissue as compared with an equivalent untreated control; such reduction or degree of prevention is at least 5%, 10%, 20%, 40%, 50%, 60%, 80%, 90%, 95%, or 100% as measured by any standard technique. According to this invention, inflammation may be measured, for example, by the detection of infiltrating leucocytes (e.g., by immunohistochemistry), the release of pro-inflammatory molecules (e.g., MCP-1), or the activation of inflammatory signaling pathway (e.g., activation of the NF-κB pathway). Thus, the treatment, reduction, or prevention of cardiac inflammation may also be measured by the ability to reduce the activation of inflammatory signaling pathways in a cardiomyocyte as measured by any standard technique.

By “treating, reducing, or preventing ischemic-reperfusion injury” or by “treating, reducing or preventing a cardiac disorder” is meant treating, or ameliorating such injury or cardiac disorder, respectively, before or after it has occurred. As compared with an equivalent untreated control, such reduction or degree of prevention is at least 5%, 10%, 20%, 40%, 50%, 60%, 80%, 90%, 95%, or 100% as measured by any standard technique known in the art.

By “dominant negative FADD” or “dnFADD” is meant any polypeptide having at least 50%, 70%, 80%, 90%, 95%, or even 99% sequence identity to 10, 20, 35, 50, 100, 150, or more than 150 amino acids of the wild type human FADD protein. In addition to inactivating mutations, such as those described herein, FADD-dn may consist of deletions or truncations of a wild-type FADD molecule. For example, a dnFADD may be a truncated FADD mutant that has a deletion of the N-terminal DED of FADD and that binds the cytoplasmic death domains of DR but cannot activate caspase-8.

By “an effective amount” is meant an amount of a compound, alone or in a combination according to the invention, required to reduce or prevent cardiomyocyte apoptosis, or alternatively, to treat, reduce, or prevent cardiac inflammation, ischemic injury, or a cardiac disorder. The effective amount of active compound(s) varies depending upon the route of administration, the age, body weight, and general health of the subject. Ultimately, the attending physician or veterinarian will decide the appropriate amount and dosage regimen.

By “acute” is meant a condition having a short course (for example, less than weeks or months), often sudden onset, and resulting from a disease process.

By a “candidate compound” is meant a chemical, be it naturally-occurring or artificially-derived, that is tested for its ability to reduce FADD expression or activity. Candidate compounds may include, for example, peptides, polypeptides, synthetic organic molecules, naturally-occurring organic molecules, nucleic acid molecules, and components thereof.

By “FADD fusion gene” is meant an FADD promoter and/or all or part of a FADD coding region operably linked to a second, heterologous nucleic acid sequence. In preferred embodiments, the second, heterologous nucleic acid sequence is a reporter gene, that is, a gene whose expression may be assayed; reporter genes include, without limitation, those encoding glucuronidase (GUS), luciferase, chloramphenicol transacetylase (CAT), green fluorescent protein (GFP), alkaline phosphatase, and β-galactosidase.

By “iatrogenically-induced” is meant a condition that is of longer duration than acute, and is planned, or is a consequence of a medical treatment (for example, a surgical technique).

The present invention provides significant advantages over standard therapies for the treatment or prevention of cardiac disorders resulting from chronic ischemic injury, acute ischemic injury, ischemia-reperfusion injury, myocardial infarction, myocarditis, heart failure, cardiac transplantation, or autoimmune disorders. Inhibition of FADD according to the present invention prevents apoptosis of cardiomyocytes, cardiac inflammation, or both. The fact that the methods of the present invention allow the simultaneous reduction in apoptosis of cardiomyocytes and cardiac inflammation differs significantly from currently available methods which tend to improve one of these conditions while exacerbating the other. In addition, the candidate compound screening methods provided by this invention allow for the identification of novel therapeutics that modify the injury process, rather than merely mitigating the symptoms.

### BRIEF DESCRIPTION OF DRAWINGS

FIG. 1 is a diagram showing the mechanisms underlying apoptosis.

FIG. 2A is a series of immunoblots showing caspase-3 activation in rat neonatal cardiomyocytes in hypoxia and Serum-Deprivation (SD) conditions.

FIG. 2B is a graph showing activity of caspase-3 over time in cardiomyocytes in normoxia or hypoxia/SD conditions.

FIG. 2C is an immunoblot showing caspase-3 activation and a picture of DNA laddering gel in neonatal rat cardiomyocytes in hypoxia, SD conditions, or in hypoxia/SD conditions.

FIG. 2D is a bar graph showing levels of caspase 3-activity of neonatal rat cardiomyocytes in hypoxia, SD conditions, or both.

FIG. 3A is a graph showing DNA fragmentation in cardiomyocytes treated with increasing concentrations of zVAD-fmk, in hypoxia/SD or normoxic conditions.

FIG. 3B is a picture showing DNA laddering in cardiomyocytes treated with increasing concentrations of zVAD-fink, in hypoxia/SD or normoxic conditions.

FIG. 3C is a graph showing caspase-8 activity over time in neonatal rat cardiomyocytes exposed to hypoxia/SD or normoxia.

FIG. 4A is an immunoblot showing the expression of FADD-dn in uninfected cardiomyocytes and cardiomyocytes infected with Ad.GFP or AdFADD-dn.

FIG. 4B is a graph showing DNA fragmentation over time, as measured by cell death ELISA of cardiomyocytes infected either with Ad.GFP or Ad.FADD-dn.

FIG. 4C is a series of gel pictures showing DNA laddering in cardiomyocytes infected with Ad.GFP, Ad.IGF-1, Ad.FADD-wt, or Ad.FADD-dn under hypoxia/SD or normoxic conditions.

FIG. 4D is a bar graph showing DNA fragmentation as measured by cell death ELISA, of cardiomyocytes infected with Ad.GFP, Ad.FADD-wt, or Ad.FADD-dn, in normoxic or hypoxia/SD conditions.

FIG. 4E is a bar graph showing caspase-8 activity in cardiomyocytes treated with zIETD-fink, in normoxic or hypoxia/SD condtions.

FIG. 5A is a bar graph showing caspase-3 activity in uninfected cardiomyocytes or cardiomyocytes infected with Ad.GFP, Ad.FADD-wt, or Ad.FADD-dn, in the presence of normoxia or hypoxia/SD.

FIG. 5B is a series of immunoblots showing caspase-3 activity, protein expression of FADD-wt, FADD-dn, or β-actin in uninfected cardiomyocytes or cardiomyocytes infected with Ad.GFP, Ad.FADD-wt, or Ad.FADD-dn, in the presence of normoxia or hypoxia/SD.

FIG. 6A is a bar graph showing caspase-8 activity in cardiomyocytes infected with AD.GFP or Ad.FADD-dn, in normoxia or hypoxia/SD.

FIG. 6B is a bar graph showing caspase-9 activity in uninfected cardiomyocytes in the presence or absence of zIETD-fink and in cardiomyocytes infected with Ad.GFP or Ad.FADD-dn. All four groups of cardiomyocytes are either in normoxia or Hypoxia/SD.

FIG. 7 is a picture of an electomobility shift assay (EMSA) showing the DNA binding activity of nuclear NF-κB subunits. A “Super-shift” assay is also performed to show that p65 is an important constituent of nuclear NF-κB-binding activity.

FIG. 8A is an immunoblot showing p65 nuclear translocation in cultured neonatal rat CM infected with Ad.GFP or Ad.FADD in the presence or absence of TNF-α (50 ng/ml). Oct-1 (control nuclear protein) and FADD expression are also shown.

FIG. 8B is a series of confocal microscopy pictures showing p65 localization in rat cardiomyocytes infected with viral constructs encoding GFP or FADD. Cells were serum-deprived for 2 hours prior to stimulation with TNF-α (50 ng/ml). p65 NF-κB immunostaining was visualized by confocal microscopy in Ad.FADD and control virus-infected CM, with or without TNF- [symbol] stimulation. Green: GFP. Red: p65. Right column represents merge of GFP and p65 staining images. Scale bar=20 μm.

FIG. 8C is a picture representing an Electrophoretic Mobility Shift Assay (EMSA. Nuclear extracts from cells infected with the indicated virus with or without TNF-α stimulation were assayed for NF-κB binding activity.

FIGS. 8D and 8E are graphs representing a Quantitative RT-PCR analysis of VCAM-1 mRNA in CM. Cells were infected and treated as above.

FIG. 8D shows representative amplification plots. FIG. 8E shows cumulative data from all 8 independent experiments with each condition tested 5-8 times. *p<0.01; **p=0.08.

FIG. 9A is a picture representing an EMSA of NF-κB activity. Human umbilical vein endothelial cells (HUVEC) were incubated with the indicated viral constructs overnight and transgene expression was confirmed by Western blotting. HUVEC expressing GFP, FADD, or FADD-ΔDED were either not treated or treated with TNF- [symbol] (50 ng/ml) or LPS (100 ng/ml) for 30 min. Nuclear extracts were assayed for NF-κB binding activity using an EMSA.

FIG. 9B is a gel picture showing NF-κB activation in Rat pulmonary artery smooth muscle cells (rPASMCs). Neither FADD nor FADD-ΔDED expression affected NF-κB activation at baseline or in response to TNF- [symbol] in rPASMCs. IKKβ-dn, in contrast, inhibited both baseline and TNF-α induced NF-κB DNA binding activity.

FIG. 9C is a picture of a gel showing nuclear p65 expression in HEK 293 cells. Cells were uninfected (lanes 1-2) or infected with GFP or FADD constructs overnight (lanes 3-4). FADD expression increased nuclear p65 in a manner comparable to that seen with TNF- [symbol] stimulation.

FIG. 9D is a gel picture showing NF-κB activation in HEK cells. Consistent with data obtained above, FADD increased nuclear NF-κB DNA binding activity in the absence of cytokine stimulation. 7.5 μg nuclear proteins were used in lanes 1-2 and 15 μg proteins used in lanes 3-4.

FIG. 10 is a series of immunoblots showing that FADD inhibits TNF-α-induced phosphorylation of IκB- [symbol] in rat neonatal CM. Cells were infected with Ad.GFP or Ad.FADD overnight followed by serum-deprivation for 2 hours. TNF- [symbol] -induced IκB- [symbol] phosphorylation was evident as early as five minutes after stimulation in Ad.GFP infected CM. Neither GFP nor FADD expression induced detectable phosphorylation of IκB-α in the absence of TNF- [symbol] stimulation. However, FADD expression inhibited TNF-α-induced IκB- [symbol] phosphorylation.

FIGS. 11A and 11B are a series of pictures showing that FADD inhibits NIK- or IKKβ-mediated NF-κB activation. Rat neonatal CM were infected with NIK or IKK [symbol] viruses in combination with either Ad.GFP or Ad.FADD. Cells were serum-deprived for 2 hours and phosphorylation of IKK [symbol] and IκB- [symbol] was detected by immunoblotting. FADD expression inhibited IκB- [symbol] phosphorylation (11A, top panel) and NF-κB binding activity (11B) to both NIK and IKKβ expression, as well as IKK phosphorylation to IKK [symbol] expression (11A, second panel from top). The endogenous rat IKK [symbol] is not recognized by the available antibodies and thus IKK [symbol] phosphorylation cannot be assessed in NIK-transduced CM.

FIGS. 12A and 12B are immunoblots showing expression of cytosolic IκB- [symbol] and NF-κB p65 subunit in cardiac tissue of mice treated with Ad.EGF.β-gal and Ad.dn-IKK-β before and following ischemia-reperfusion injury.

FIG. 12C is a series of confocal microscopy images identifying subcellular localization of p65 in the myocardium.

FIG. 13 is a series of graphs showing the mRNA levels of ICAM-1, VCAM-1, MCP-1 as measured by quantitative RT-PCR in cardiomyocytes treated with or without TNF-α.

FIG. 14 is a series of graphs showing upregulation of ICAM-1 and VCAM-1 mRNA in cardiomyocytes of rats subjected to sham or ischemia operation, as measured by quantitative RT-PCR.

FIG. 15A is an immunoblot showing the expression of FADD-wt, FADD-dn and GFP in rats treated with Ad.GFP or Ad.FADD-dn.

FIG. 15B is a picture of the infarct of rats treated with Ad.GFP or Ad.FADD-dn.

FIG. 15C is a bar graph showing the percentage of myocardial infarction (measured as a % AAR) in rats treated with saline, Ad.GFP, or Ad.FADD-dn.

### DETAILED DESCRIPTION

Apoptosis and inflammation can both contribute to cardiac injury after transient ischemia. Unfortunately, many interventions that mitigate either one of these processes tend to exacerbate the other. Our results demonstrate that signaling via the Death Receptor (DR) adaptor protein, Fas-associated death domain protein (FADD), is critical to both cardiomyocyte survival during hypoxia and cardiac inflammation by virtue of its inhibition of NF-κB activation. In particular, the inhibition of FADD by the expression of a dominant negative FADD has the unusual effect of simultaneously promoting cardiomyocyte survival and inhibiting cardiac inflammation by inhibition of NF-κB activation. Overall, inhibiting FADD using an FADD inhibitor that is anti-apoptotic, anti-inflammatory, or both can mediate substantial benefits in cardiac disorders that are sustained over time.

The Role of FADD in Apoptosis

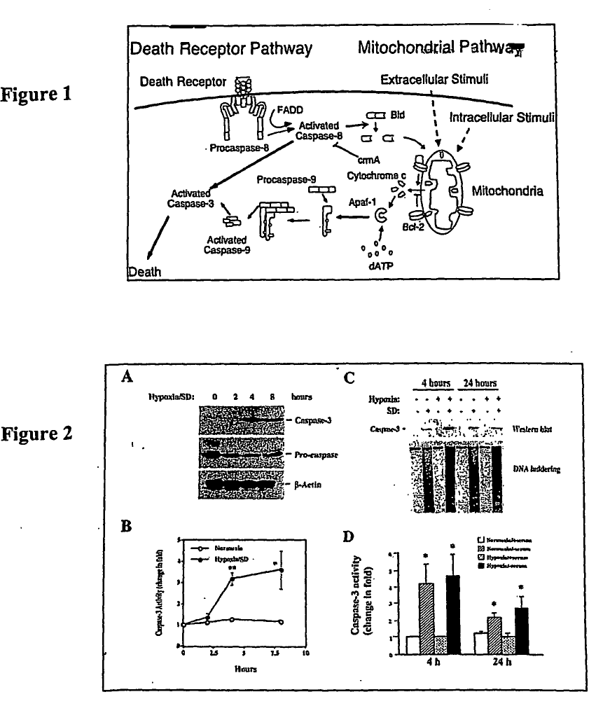

Although a fully comprehensive model of apoptosis has not yet been assembled, two general pathways have been delineated (FIG. 1). In the extrinsic pathway, engagement of “Death Receptors (DR)” (e.g., TNFR60, Fas/Apo1, or TRAIL-R/Apo2) leads to the recruitment of specific adaptor molecules, such as Fas-associated death domain protein (FADD), which bind to DR via homologous death domains (DD) present in both molecules. The other end of FADD contains additional “death effector domains” (DED) that bind homologous DEDs in caspase-8 and FLIP (Flice Inhibitory Protein), a potent inhibitor of caspase-8. Following the activation of caspase-8, the effector caspase-3 is cleaved and activated, ultimately resulting in cell death. In addition to their role in classical ligand induced receptor activation, DR are also involved in cell death settings mediated by external stimuli such as UV irradiation, chemotherapy, or cell detachment, where ligand-independent DR activation can occur.

The intrinsic pathway or Mitochondria Pathway (MP) of apoptosis occurs when specific stimuli lead to the mitochondrial release of cytochrome c (cyt c) and other factors, usually in association with alterations of mitochondrial membrane potential. Cyt c binds Apaf-1 in the cytosol, induces its oligomerization and the subsequent recruitment of procaspase-9. This culminates in the activation of caspase-9, which can subsequently cleave and activate effector caspases, such as caspase-3. In turn, these effector caspases dismantle the cellular components by cleaving a wide variety of non-caspase cellular proteins. The precise functional contribution of each substrate to programmed cell death remains poorly defined but some targets, such as poly (ADP-ribose) polymerase (PARP), at the very least, provide a useful index of caspase-mediated apoptosis. Along with cyt c, the mitochondria release Smac/DIABLO which plays a critical role in some cells by releasing caspase-3 from the inhibition mediated by IAPs (inhibitors of apoptosis). The pathways leading to mitochondrial release of cyt c/Smac are less well delineated than DR signaling. However, it classically occurs in cells deprived of specific growth factors or critical metabolic substrates.

In the context of cardiomyocytes, CM apoptosis has been identified in many clinical settings including congestive heart failure, transplanted allografts and ischemic reperfusion injuries. Experimental evidence suggests that both DR- and MP-induced apoptosis occur in CM. In the complex setting of CM hypoxia or ischemia, it seems likely that there would be contributions from both of these pathways and that interventions blocking one or the other might simply channel CM death through the alternative apoptotic pathway, or even necrosis. Surprisingly, our results suggest that DR-signaling via FADD plays a dominant role in hypoxia- and serum-deprivation-induced CM apoptosis, acting upstream of both caspase-8 and caspase-9.

The Role of FADD in Inflammation

Inflammatory signaling contributes to a variety of cardiac conditions including injury after transient ischemia, myocarditis, and heart failure. In addition to its role in apoptosis, Fas/FADD signaling also plays a role in cell proliferation, differentiation, and inflammation in some systems. In the heart, for example, transgenic expression of the FasL induces inflammation. In vascular smooth muscle cells, signaling through the Fas/FADD pathway triggers a program of pro-inflammatory gene expression concomitant with NF-κB activation thereby enhancing the production of proinflammatory cytokines such as monocyte-chemoattractant protein 1 (MCP-1) and IL-8 (Wajant et al., (2000) J Biol Chem 275: 24357-24366; Hu et al., (2000) J Biol Chem 275:10838-10844; Chaudhary et al., (2000) Oncogene 19:4451-4460; Schaub et al., (2000) Nat Med 6:790-796), and may in turn contribute to the pathogenesis of vascular diseases (Schaub et al. (2000) Nature Medicine; 6: 790-6.). Furthermore, FADD can also activate NF-κB in a variety of other cell types including HeLa cells and 293 cells.

The nuclear factor kappa B (NF-κB) family of transcription factors is activated by diverse stimuli including oxidative stress and inflammatory cytokines. Their expression drives the transcription of many genes involved in inflammation and cell survival, both prominent components of IRI and MI, as noted above. For example, as a result of the expression of NF-κB early on in IRI, cytokines and adhesion molecules are expressed and enhance the local recruitment of inflammatory leukocytes that may contribute to IR injury.

NF-κB factors (p65 or RelA, p50, p52, BC1-3, c-Rel, and RelB) generally exist as dimers in the cytoplasm bound to an inhibitory subunit, IκB (Collins et al., (2001) J Clin Invest 107: 255-264). A dominant mechanism of NF-κB activation involves serine phosphorylation and degradation of IκB via the ubiquitin pathway, followed by translocation of NF-κB to the nucleus where it activates transcription of specific promoter targets. This serine phosphorylation is mediated by a large multi-unit complex containing two catalytic subunits (IKK- [symbol] and IKK-β), as well as the regulatory subunit IKK-γ or NEMO (Collins et al, supra). IKK- [symbol] appears to be the major kinase responsible for phosphorylation of all three (α, β, and ε) IκB subunits (DiDonato et al. (1997)

Nature 388: 548-554; Mercurio et al., (1997) Science 278: 860-866). IKK-β itself undergoes phosphorylation that appears mediated through trans-autophosphorylation brought about by induced proximity (Ghosh et al., (2002) Cell 109: S81-96). Although not essential for IKK-β activation by TNF [symbol] (Yin et al (2001) Science 291:2162-2165), the kinase, NIK, can also activate IKK-β and NF-κB.

Although inhibition of NF-κB may attenuate cardiac injury by minimizing inflammation in the heart, NF-κB also drives the expression of survival factors that appear particularly important in cardiomyocytes. Such factors include, for example, the Inhibitors of Apoptosis (IAPs), which inhibit caspase-3 and in some settings must be removed by mitochondrial Smac release for DR-induced apoptosis. In cardiomyocytes, downstream inhibition of NF-κB through expression of either a transdominant IκB or dominant negative IKK-βpotentiates apoptosis (Mustapha et al., (2000) Am J Physiol Heart Circ Physiol 279:H939-945; Cook et al., (2003) Circulation 108:664-667) raising concerns that therapeutic strategies directed at NF-κB inhibition could have adverse effects on the heart. Thus, while NF-κB inhibition might limit inflammation in IRI, it could also potentiate cardiomyocyte apoptosis. In this respect, most interventions that inhibit NF-κB also potentiate apoptosis in both CM and endothelial cells and consequently, minimizing one aspect of cardiac injury would exacerbate the other.

In sharp contrast to previous experimental evidence, our data in CM demonstrates an unusual inhibition of NF-κB signaling by both FADD and FADD-dn (the truncation mutant lacking the DED (ΔDED)), most likely through alternative DED-independent mechanisms. Thus, our results showing that FADD-dn can block both NF-κB activation in response to inflammation and apoptosis are unexpected. We further show that the regulatory effect of FADD on NF-κB signaling is cell-type specific and is mediated, at least in part, by inhibiting the phosphorylation and activation of IKKβ.

FADD Inhibitors

According to the present invention, an “anti-apoptotic FADD inhibitor” is any agent having the ability to reduce or prevent apoptosis of cardiomyocytes by at least 10%, 20%, 30%, 40%, 50%, 60%, 70%, 80%, 90%, or 100% relative to an untreated control cell. Such reduction or prevention in apoptosis may be measured by any technique known in the art including, for example, measurements of apoptosis in vitro (as measured, for example, by DNA laddering, TUNEL assay, Flow cytometry for DNA content, cell death ELISA, caspase activity, or detection of surrogate markers of apoptosis by Western or Northern analysis) or in vivo (as measured by in situ TUNEL, DNA laddering, or detection of surrogate markers of apoptosis by immunohistochemistry).

An “anti-inflammatory FADD inhibitor” of the invention is any compound having the ability to treat, reduce, or prevent cardiac inflammation by at least 10%, 20%, 30%, 40%, 50%, 60%, 70%, 80%, 90%, or 100% relative to an untreated control. It is understood that cardiac inflammation can be measured by any standard method in the art used for the assessment of inflammation (e.g., detection of pro-inflammatory markers such as NF-κB for example, morphology, and detection of infiltration of inflammatory cells such as leukocytes by immunohistochemical methods). According to this invention, an FADD protein may also be administered as an anti-inflammatory agent.

An anti-apoptotic FADD inhibitor may or may not be have anti-inflammatory activities and similarly, an anti-inflammatory FADD inhibitor may or may not be an anti-apoptotic FADD inhibitor. The FADD inhibitor (anti-apoptotic, anti-inflammatory, or both) of the invention can be, for example, a dominant negative FADD protein. A dominant negative FADD protein is any amino acid molecule having a sequence that has at least 50%, 70%, 80%, 90%, 95%, or even 99% sequence identity to at least 10, 20, 35, 50, 100, or more than 150 amino acids of the wild type human FADD protein. For example, a dominant-negative FADD protein may have a deletion of the N-terminal DED of FADD such that it binds the cytoplasmic death domains of DR but cannot activate caspase-8.

According to this invention, the anti-apoptotic or anti-inflammatory FADD inhibitor may be administered as an expression vector. The expression vector may be a non-viral vector, or a viral vector (e.g., retrovirus, recombinant adeno-associated virus, or a recombinant adenoviral vector). Alternatively, the anti-inflammatory or anti-apoptotic FADD inhibitor may be directly administered as a recombinant protein to cardiomyocytes using for example microinjection techniques. Optionally, an FADD inhibitor may be a small molecule antagonist, or an antisense to FADD. RNA interference (RNAi) may also be used to target FADD as it provides a powerful method of gene silencing in eukaryotic cells including proliferating mammalian cells such as the cardiomyocytes of the present invention. The basic technique of RNAi involves introducing sequence-specific double-stranded RNA into cardiomyocytes in order to generate a nonheritable, epigenetic knockout of gene function that phenocopies a null mutation in the targeted gene. RNA interference has previously been described (O'Neil N J, et al.(2001) Am J Pharmacogenomics 1: 45-53).

Recombinant Adenoviral Vectors

Three recombinant type 5 adenoviruses (Ad.EGFP/β-gal, Ad.FADD-wt and Ad.FADD-dn) were used in these studies. Ad.EGFP.β-galactosidase (Ad.EGFP/β-gal) has been described in detail by Matsui et al. (Circulation 104:330-335, 2001). Ad.FADD-wt and Ad.FADD-dn were both constructed by subcloning the cDNA encoding the wild type FADD or the FADD protein having a deletion of the N-terminal DED of FADD, respectively, with a carboxy-terminal Flag epitope into the shuttle plasmid, pAdTrack-CMV, which also encodes a separate expression cassette for CMV-driven EGFP expression. Full length adenoviral DNA clones, incorporating this shuttle vector, were obtained through homologous recombination with pAdEasy-1 in E. coli (BJ5183) and prepared as high titer stocks, as described by He et al. (Proc. Natl. Acad. Sci. USA (1998) 95:2509-14). Adenoviral vectors were amplified in 293 cells, particle count estimated from OD₂₆₀ and titer determined by plaque assay. Stock titers were >10⁹ pfu/ml for each vector with a particle/pfu ratio of about 20-50. Vector doses are expressed as multiplicity of infection (MOI), defined as plaque-forming units per cell. Wild-type adenovirus contamination was excluded by the absence of PCR-detectable E1 sequences.

The adenoviral vector that encodes dominant negative IKKβ (Ad.IKKβ-dn) has been described previously in detail (Chao et al., (2002) J Biol Chem 277: 31639-31645; Meiler et al., (2002) J Mol Cell Cardiol 34: 349-359). Ad.NIK and Ad.IKKβ were constructed in a similar manner to Ad.FADD and encode wild-type forms of these kinases. Viral titer was determined by plaque assay in 293 cells. Stock titers were >1010 pfu/ml for each vector with a particle/pfu ratio=10-100. Wild-type adenovirus contamination was excluded by the absence of PCR-detectable E1 sequences.

Source of Cardiomyocytes

Isolated cardiomyocyte cultures or cardiomyocytes present within cardiac tissue or the heart organ may be treated with an FADD inhibitor of the present invention. In addition to human, a cardiomyocyte according to the methods of the present invention may be derived from any mammal, including for example, a pig, mouse, or non-human primate monkey. Cardiomyocytes amenable to treatment may be of any maturity state, and thus include neonatal cardiomyocytes, stem cells, cells committed to differentiate to cardiomyocytes, a myocyte derived from a non-heart muscle, a myoblast, or adult cardiomyocytes. Mammals having a cardiac disorder may be directly administered with an FADD inhibitor. Alternatively, cardiomyocytes, or cardiac tissue may be isolated from a mammal, treated with an FADD inhibitor ex vivo and transplanted back into the patient. Such cardiomyocytes, cardiac tissue, or heart may also derive from a donor mammal for transplantation into a recipient mammal. The donor and recipient may or may not be from the same species. For example, a pig heart treated according to the methods of this invention may be transplanted into a human recipient.

In Vitro Model of Neonatal Rat Cardiomyocyte

Cardiomyocytes (CM) were prepared from 1-2 day-old rats as described by Matsui et al. (Circulation 100:2373-9, 1999). All CM were incubated in RPMI-1640 containing 5% FBS and 10% horse serum. Neonatal CM grown in 60 mm dishes were infected with Ad.EGFP.β-gal (MOI 20), Ad.FADD-wt, or Ad.FADD-dn (MOI 20), for 24 hours prior to exposure to transient hypoxic conditions, lasting up to 24 hours. To expose CM to transient hypoxia conditions, CM media is changed to N₂/CO₂-saturated serum-free F10 and cells are placed in an airtight N₂-saturated container at 37° C. for up to 24 hours. Hypoxia was confirmed by oxymetry measurements. To examine the role of FADD in inflammatory signaling, cultured neonatal rat CM were stimulated with TNF-α (50 ng/ml) for 4 hours. Nuclear and cytoplasmic extracts were prepared.

Cell Cultures

293 cells were cultured in Dulbecco's modified Eagle's medium supplemented with 10% horse serum and 5% fetal bovine serum. Rat pulmonary artery smooth muscle cells (rPASMCs) were prepared and incubated in RPMI-1640 containing 5% FBS and 10% horse serum, penicillin and streptomycin (Takata et al., (2001) Am J Physiol Lung Cell Mol Physiol 280:L272-278). rPASMCs were used between passages 3-10. Human umbilical vein endothelial cells (HUVEC) were isolated and cultured in M199 with 20% fetal bovine serum, endothelial cell growth factor, porcine intestinal heparin (50 μg/ml), and antibodies (Gerszten et al., (2001) J Biol Chem 276:26846-26851). For gene transfer, cells were either left uninfected or infected with the indicated adenoviral constructs overnight in regular culture media. Transgene expression was confirmed by immunoblotting. For TNF-α and LPS stimulation, cells were serum-deprived for 2 hours prior to the treatments.

In Vitro Apoptosis Assays

A number of assays may be used to measure cardiomyocyte apoptosis in vitro, including for example DNA laddering, cell death ELISA, flow cytometry for DNA content, TUNEL assay, the measurement of caspase activity, or the detection of surrogate markers of apoptosis by Western or Northern analysis, or alternatively by RT-PCR.

For determination of DNA laddering, cellular DNA is extracted with phenol:chloroform, treated with RNase, ³²P-labeled, and then visualized by electrophoresis in 1.8% agarose gel.

In the TUNEL assay, terminal deoxynucleotidyl transferase is used to incorporate digoxigenin-labeled dUTP into 3′-OH DNA ends generated by DNA fragmentation and detected by counterstaining with peroxidase labeled anti-digoxigenin mAb (ApoTag, Intergen).

Flow cytometry may also be used to measure DNA content in cardiomyocytes. Cells were fixed with 80% ethanol and stained with propidium iodide after RNase treatment. Apoptotic cells register as containing less than the diploid DNA quantity (2N).

Histone-associated DNA fragments were quantified by cell death ELISA as described in the manufacturer's protocol. DNA fragmentation data were corrected for background and normalized to the result with normoxic cardiomyocytes.

Optionally, apoptosis may also be measured by the protein or gene expression, or alternatively, by the activity of surrogate markers of apoptosis including, for example, caspase activity. Caspase-3, -8, and -9 activity may be examined by using the caspase colorimetric assay kit from R&D Systems according to the manufacturer's protocol. Briefly, cells are scraped, collected, washed with cold PBS, and lysed in cold lysis buffer. Lysates are incubated on ice for 10 min and centrifuged (10,000×g, 1 min). The supernatants are removed and assayed for caspase activity. The specific peptide substrates used for each individual caspase are DEAD-pNA, LEHD-pNA, and IETD-pNA for caspase-3, -9, and -8, respectively. Release of the pNA cleavage product is quantitated in a microplate reader (Bio-Rad) at a wavelength of 405 nm.

Optionally, several overlapping assays may be performed to ensure that cell death is apoptotic (nuclear morphology and DNA laddering), that dying cells are in fact CM (TUNEL/double staining/confocal), and that quantitative comparisons of different populations can be made (ELISA for histone-associated DNA fragments/FACS for DNA content). General assays of cell viability (e.g. trypan blue exclusion, MTT, etc.) can also be used to assess overall cytoprotection (i.e. not just protection from apoptosis).

Animal Studies

Sprague-Dawley rats weighing 200-300 grams and were subjected to in vivo gene transfer with Ad.EGFP.β-gal, Ad.FADD-wt, or Ad.FADD-dn. Forty-eight hours after adenoviral infection, animals were subjected to either 30 min of ischemia followed by the indicated period of reperfusion (IRI), no reperfusion (MI), or to sham operation. For the transfer of a cardiac gene in vivo, males were anesthetized (pentobarbital), intubated, and ventilated (SAR-830, CWE Inc.) after which, 200 μl of solution containing 1.2×10¹² particles/ml Adenovirus (Ad.EGFP.β-gal, Ad.FADD-wt, or Ad.FADD-dn) or buffer alone were injected via left thoracotomy into the antero-apical myocardium. To induce ischemic reperfusion, a left thoracotomy was performed and LAD ligated with silk suture (5.0) ˜4 mm from its origin with a slipknot. Ischemia was confirmed by myocardial blanching and EKG evidence of injury. Fluorescent microspheres (300 μl; 10 μm FluoSpheres, Molecular Probes) were injected into the LV cavity five minutes after induction of ischemia. For IRI, the LAD ligature was released after 30 min, and reperfusion visually confirmed. For infarction, the LAD ligature was permanently tied. In the sham-operated animals, a suture was placed under the LCA but was not tied. Animals were anesthetized and exsanguinated while the heart was perfusion-fixed in situ with 4% paraformaldehyde at a constant pressure of 100 cm H₂O for 20 min or removed and cryopreserved for immunohistochemistry, DNA, and protein analysis. Blood sampling was also used for determination of routine chemistries (including CPK, LDH, SGOT, and glucose), using commercially available kits. Hearts were frozen in liquid N₂, and sectioned from apex to base (Jung Frigocut 2800E, Leica) into four 2 mm sections, each separated by six 10 μm sections. Thicker sections were used to quantify the area-at-risk (AAR) and the infarct area. For % MI, sections were incubated in 5% (w/v) triphenyltetrazolium chloride (TTC, Sigma) in PBS (pH 7.4) at 37° C. for 20 min. For each section, the AAR and infarct area were measured from enlarged digital micrographs using NIH image. The % MI was calculated as the total infarction area divided by the total AAR for that heart.

Determination of Apoptosis In Vivo

To determine apoptosis in vivo, TUNEL staining was performed using Apoptag (Intergen) according to the manufacturer's instructions, with Hoechst 33258 (Sigma) nuclear counter-staining. Nuclei were counted in 8-10 microscope fields from a 10 μm mid-ventricular section for each heart used to assess infarct size as described previously by Matsui et al. (Circulation (2001) 104: 330-335). Alternatively, DNA laddering was also used to detect apoptosis of cardiomyocytes. Fresh tissues (without TTC staining) were microdissected under UV light into ischemic and non-ischemic regions and processed simultaneously. Tissues from each region were lysed (100 mM Tris [pH 8.5], 5 mM EDTA, 0.2% SDS, 200 mM NaCl, 100 μg/ml proteinase-K) at 37° C. for 18-20 hr. DNA was prepared, labeled with [α-³²P] dCTP, and subjected to electrophoresis and autoradiography as described by Vazquez-Jimenez et al (J. Am. Coll. Cardiol., 2001). Optionally, apoptosis of cardiomyocytes may further be determined by the detection of surrogate markers of apoptosis, such caspase activation, for example.

Determination of Inflammation

Cardiac inflammation may be determined by the detection of pro-inflammatory markers, the release of pro-inflammatory molecules (MCP-1), or by the activation of pro-inflammatory signaling in cardiomyocytes. NF-κB activation correlates with cardiac inflammation and may be determined both in vitro (by Western or Northern analysis for example) or in vivo (as measured by immunohistochemical methods). NF-κB activation is typically demonstrated by phosphorylation and degradation of IκB, nuclear translocation of the p65 NF-κB subunit, or increased mRNA for the NF-κB-dependent transcripts, VCAM-1 and ICAM-1. Alternatively, overall morphology of cardiac tissue and the detection of infiltration of inflammatory cells, such as leukocytes (by immunohistochemical methods) also demonstrate the presence of inflammation in cardiac tissue.

Determination of Leukocyte Infiltration

Leukocytes were quantitated by counting immunofluorescent cells on frozen sections using a monoclonal antibody to rat leukocyte populations that are available from Sera-Lab (Accurate Chemicals, Westbury, N.Y.) for total leukocytes (CD45, OX-1), monocytes (ED-1), T cells (TCR, R73 and CD2, OX-34), CD4+ T cells (CD4, W3/35), CD8+ T cells (CD8, OX-8), and granulocytes. PMN infiltration may also be evaluated by MPO as described previously by Matsui et al (Circulation 104:330-335, 2001). Myocardial MPO activity was determined as an index of neutrophil infiltration. Frozen normal, ischemic, and non-ischemic heart samples (20 mg) were homogenized in 50 mmol/L potassium phosphate buffer (PPB). After centrifugation (12,500×g, 20 minutes, 4° C.), pellets were resuspended in PPB containing 0.5% hexadecyltrimethyl ammonium bromide (HTAB) (Sigma). Samples were sonicated on ice, freeze-thawed, and centrifuged (12,500×g, 20 minutes, 4° C.). Supernatants were collected and incubated with reaction buffer (0.167 mg/mL of o-dianisidine dihydrochloride, 0.0005% H₂O₂, 50 mM PPB). Absorbance was measured spectrophotometrically at a wavelength of 470 nm. MPO activity was expressed as OD_{(sample-blank)}/mg protein/minute.

Multiple (≧10) sections are tallied from each segment and numbers are tracked according regional distribution and relationship to ischemia. Adenoviral DNA, mRNA, and protein levels are examined by real-time PCR, using small amounts of tissue processed using Tri-Reagent, which simultaneously prepares DNA, RNA, and protein from the same sample, thus allowing correlation between the different assays.

Analysis of NF-κB Activation In Vitro

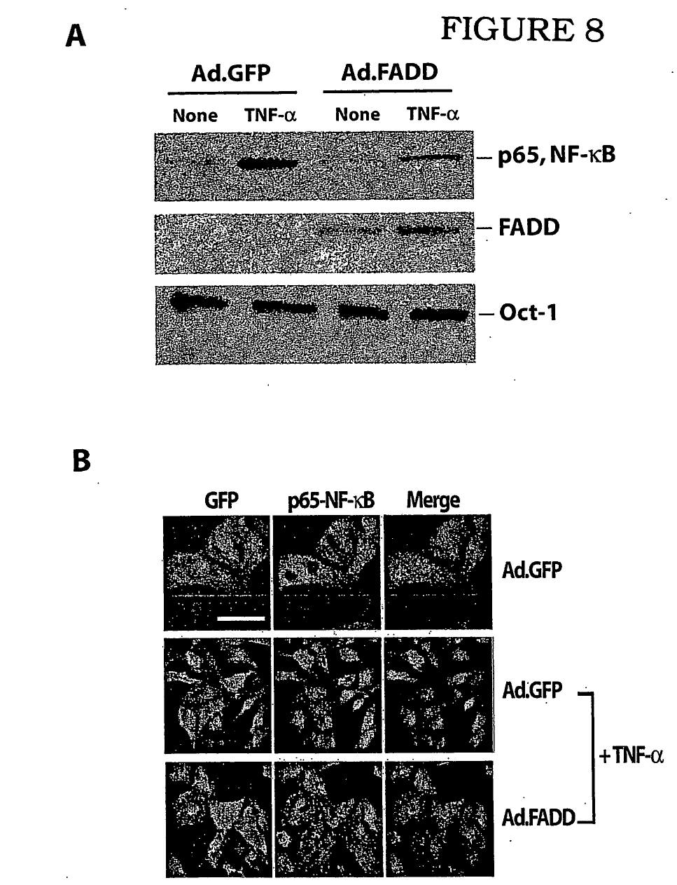

We have established a variety of techniques for analysis of NF-κB activation using commercially available reagents (described in Meiler et al. J. Mol. Cell Cardiol. 2002). These include analysis of nuclear translocation of NF-κB subunits by immunoblotting and immunohistochemistry, analysis of cytosolic degradation of IκB subunits α, [symbol] and ε, as well as phosphorylation of IκB-α and p65. In addition, we have successfully utilized commercially available NF-κB-dependent reporter plasmids to directly monitor the effects of NF-κB activation on gene transcription in cultured cells. We have also used electromobility shift assays (EMSAs) in CM to document not only the presence of specific NF-κB subunits in the nucleus (seen with immunoblotting of nuclear extracts) but also the DNA binding activity of nuclear NF-κB subunits (FIG. 7). Specificity of interaction is confirmed by inhibition of binding by excess of unlabeled NF-κB consensus binding site (FIG. 7, “cold olig”) but not mutated binding site (“mutant oligo”). “Super-shift” assays (FIG. 7, right panel) enable the identification of the NF-κB subunits that are actually binding DNA in CM nuclei. Shown here, were high (FIG. 7, right panel, middle lane) and low (FIG. 7, right lane) concentration of antibody to p65. After TNF-α, p65 is an important constituent of nuclear NF-κB-binding activity (FIG. 7, right panel). We have also generated and characterized a variety of adenoviral vectors that directly activate or inhibit NF-κB signaling. These include vectors encoding a dominant negative IKK-β that effectively and specifically inhibits NF-κB activation without affecting MAPK or JNK signaling, a wild-type IKK-β, a wild-type NIK (upstream kinase capable of directly activating IKK-β), and a kinase-inactive NIK (as a control).

MCP-1 ELISA

Myocardial homogenates were suspended in PBS solution containing protease inhibitors (PMSF 1 mM, leupeptin 1 μg/mL, aprotinin 1 μg/mL) and 1% Triton-X100. After incubation (1 hour, 4° C.), extracts were centrifuged (20,000×g, 20 minutes, 4° C.) to remove cellular debris. Expression of rat MCP-1 was quantified by ELISA.

Ventricular Pressure Measurements

1.8 Fr Millar manometry catheters are inserted directly into the left ventricular cavity prior to euthanasia to permit measurement of intracavitary pressure over time. Both direct Pressure/Time and the first derivative (dP/dt) tracings are analyzed.

Eciocardiograph

We used the GE Vivid 5 with a 15L8 linear array transducer (13.0 MHz, imaging depth 10 mm) at a frame rate of 166/sec to yield transthoracic 2DE and M-mode images suitable for analysis of: 1) wall thicknesses, 2) end-systolic and end-diastolic chamber dimensions, 3) fractional shortening and 4) ejection fraction (using a modified Simpson's rule).

Immunohistochemistry and H&E

Hearts were fixed in 4% paraformaldehyde. Five micron sections were treated with 0.1% SDS and incubated with primary antibody to NF-κB p65 for 90 minutes at 37° C. Sections were rinsed in PBS and incubated with anti-mouse IgG conjugated to tetramethyl rhodamine (60 minutes, 37° C.). Confocal images were obtained using a laser confocal system. Hematoxylin and eosin (H&E) staining was performed for histomorphologic evaluation of neutrophil infiltration.

For confocal microscopy of p65, rat CM prepared and purified using Percoll density gradient centrifugation and were incubated in 35-mm dishes at low density. CM were infected with Ad.GFP or Ad.FADD and treated with TNF- [symbol] as described above. Cells were fixed and permeabilized using Cytostaining Kit from BD PharMingen (San Diego, Calif.). p65 was stained with a polyclonal antibody (Santa Cruz) (1:100) overnight at 4° C. and Alexa Fluor 546-labeled anti-rabbit IgG (1:2000) for 1 hour at room temperature.

Preparation of Nuclear and Cytosolic Extracts

2-3×10⁶ CM in 60-mm dishes were washed once with cold PBS, scraped, and transferred to 15-ml Falcon tubes. Nuclear and cytosolic fractions were prepared using Nuclear and Cytoplasmic Extraction (NE-PER) kit from Pierce (Rockford, Ill.) as described in manufacturer's manual. 100 μl of CER I was added to each sample and 50 μl of ice-cold NER added to each nuclear fraction.

Electrophoretic Mobility Shift Assays (EMSA)

Cells were harvested and nuclear extracts prepared as above. Unless stated otherwise, aliquots of the nuclear extracts (8 μg) were incubated with 0.5 ng of a radiolabeled double-stranded oligonucleotide containing the NF-κB binding sequence (Active Motif, Carlsbad, Calif.). In some reactions such as samples from rat CMs, 1.0 ng of mutated NF-κB oligonucleotide was included to block non-specific binding. Nuclear proteins and oligonucleotide were then separated with native PAGE and detected by autoradiography.

Western Blotting

Proteins were separated by SDS-PAGE performed under reducing conditions on 7.5%, 10%, and 12% separation gels with a 4% stacking gel. Proteins were transferred to nitrocellulose membranes by semi-dry blotting. Membranes were incubated with primary antibodies to FADD, or NF-κB p65 overnight at 4° C. After washing, membranes were incubated with horseradish peroxidase-conjugated secondary antibody and immunoreactive bands detected by chemiluminescence.

RNA Extraction and Purification

Cell RNA was extracted from cultured rat CM using TRIzol reagent (Invitrogen-Life Technologies). RNA was further purified using RNase mini kit (Qiagen), eluted in 50-100 μl, and quantified using Ribogreen as described in manufacturer's instructions (Molecular Probes, Eugene, Oreg.). 5-10 μg RNA was treated with DNase and stored at −80° C. for QRT-PCR analysis.

Quantitative RT-PCR

Neonatal CM were exposed to ischemia and then harvested in Trizol reagent. Samples were centrifuged (12,000×g, 10 minutes, 4° C.), supernatants were removed and vortexed (20 seconds) with an equal volume of isopropanol. Total RNA was precipitated by centrifugation (12,000×g, 10 minutes, 4° C.) and purified. Expression of the VCAM-1, ICAM-1, and MCP-1 in samples was determined using quantitative RT-PCR analysis and sequence-specific primers. Purified RNA was quantified using Ribogreen as described in manufacturer's instructions (Molecular Probes, Eugene, Oreg.). QRT-PCR analysis was performed and analyzed as described previously (Pfaffl et al., (2002) Domest Anim Endocrinol 22:91-102; Cook et al., (2002) J Biol Chem 277:22528-22533). RNA (100 ng/reaction) was reverse transcribed and the cDNA subsequently amplified. For each PCR run, a separate pair of GAPDH primers was used as an internal control to ensure equal RNA loading. RT-PCR primers were as follows: rat GAPDH: forward: 5′-ATGCCATCACT GCCACTCAG-3′ (SEQ ID NO: 1), reverse: 5′-CAGGGATGATGTTCTGGGCT-3′ (SEQ ID NO: 2); Rat VCAM-1: forward: 5′GAAGCCGGTCATGGTCAAGT-3′ (SEQ ID NO: 3); reverse: 5′-GACGGTCACCCTTGAACAGTTC-3′ (SEQ ID NO: 4).

Statistics

Data are represented as mean±SD for continuous variables. The null hypothesis is rejected at 95% confidence (i.e. p=0.05). Rates and proportions were analyzed by Mantel-Haenszel chi-square tests of general association or Fisher's exact test as appropriate. Distributions of continuous variables were checked for normality. The Student's t-test was used to compare the means of normally distributed continuous variables and the Wilcoxon Rank sum test to compare non-normally distributed continuous

Hypoxia and Serum-Deprivation (SD) Induce DNA Fragmentation and Activation of Caspases-3 & -8 in Cardiomyocytes Concomitant with Apoptosis

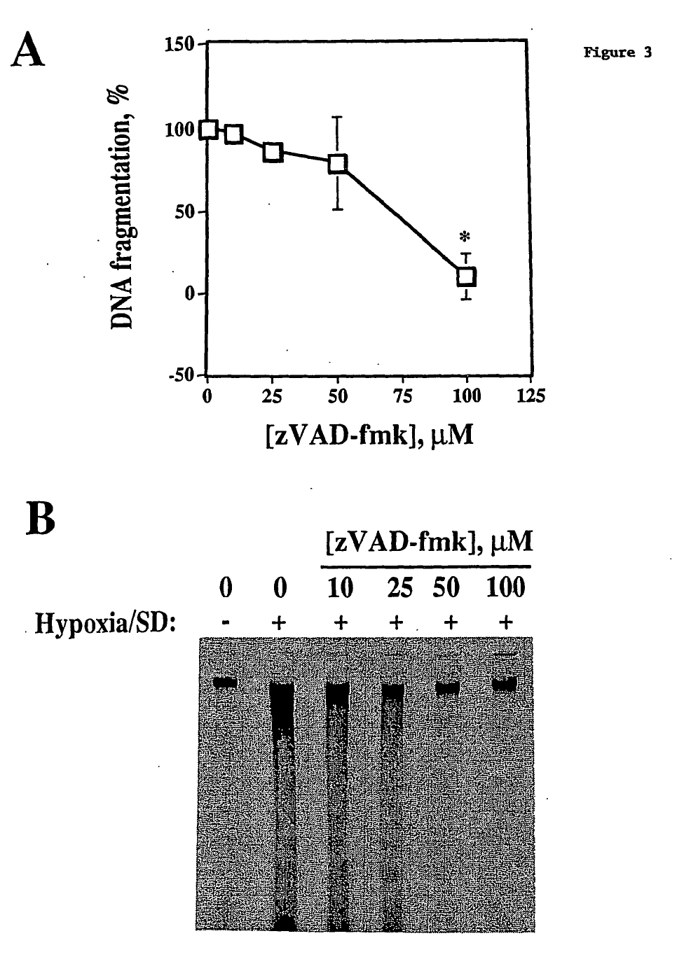

The combination of hypoxia and SD induced a time-dependent cleavage of 35 kDa pro-caspase-3 with appearance of the 19 kDa active form of caspase-3 in cardiomyocytes (FIG. 2A). The processing of procaspase-3 to caspase-3 occurred early, within 2 hours of hypoxia/SD, and reached its maximal level within four hours. The time course of pro-caspase-3 cleavage in response to hypoxia/SD corresponded well with measured caspase-3 activity, which also plateaued by four hours (FIG. 2B). To delineate the individual contribution of serum-deprivation and hypoxia to CM apoptosis, CMs were subjected to hypoxia and SD alone or in combination. As shown in FIG. 2C to FIG. 2D, within four hours of SD, there was significant DNA laddering, as well as caspase-3 cleavage and activation, even in the absence of hypoxia. The combination of hypoxia and SD modestly increased the level of caspase-3 activation (FIG. 2D) and DNA laddering at four hours (FIG. 2C), compared to SD alone. In the presence of serum, hypoxia for up to 24 hours failed to induce caspase-3 activation and DNA fragmentation (FIG. 2C to FIG. 2D). Neonatal cardiomyocytes are therefore much more sensitive to serum deprivation than to hypoxia. To define the functional importance of caspase activation in hypoxia/SD-induced CM apoptosis, we examined the effect of the general caspase inhibitor, zVAD-fmk, on hypoxia/SD-induced DNA fragmentation. Treatment of CMs with zVAD-fmk at concentrations ranging from 50 to 100 μM inhibited hypoxia/SD-induced apoptosis as evidenced by ELISA for histone-associated DNA fragmentation and by DNA laddering, confirming that this is indeed a model of caspase-dependent (apoptotic) CM death.

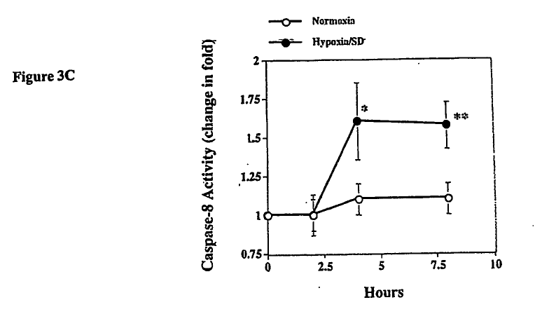

To explore the role of Death Receptor (DR) signaling in hypoxia/SD-induced CM apoptosis, we examined the activity of the apical DR caspase, caspase-8. Hypoxia/SD induced a time-dependent increase in caspase-8 activity that reached a maximal level within four hours (FIG. 3). Caspase-8 activation therefore also occurs early in hypoxia/SD and may contribute to the caspase-3 activation and overall apoptosis observed.

FADD-dn Inhibits Hypoxia/SD-Induced CM Apoptosis

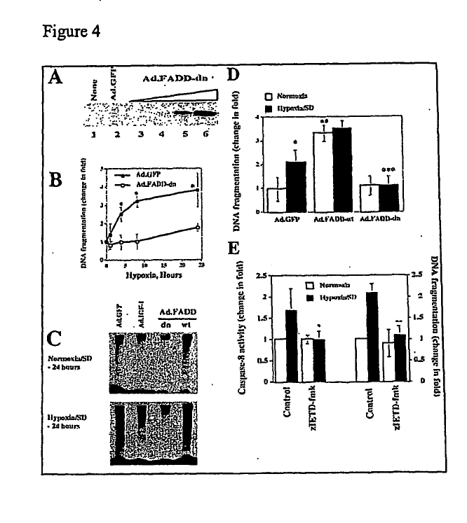

To delineate the functional contribution of DR signaling in this model, we constructed recombinant adenoviral vectors carrying the wild-type (AdFADD-wt) and dominant negative forms of FADD (AdFADD-dn) and used these vectors to transduce rat neonatal CMs subjected to hypoxia/SD conditions. As shown in FIG. 4A, adenoviral gene transfer induced dose-dependent expression of the FADD-dn protein in CM (FIG. 4A). Hypoxia and SD induced up to a 3.8-fold increase in DNA fragmentation within 24 hours in CMs infected with control virus. Adenoviral expression of FADD-dn, but not FADD-wt, dramatically reduced the apoptotic effect of hypoxia/SD as demonstrated by histone-DNA fragmentation ELISA and DNA laddering (FIG. 4). Similarly, adenoviral expression of IGF-I, which has a known cardioprotective effect, also inhibited DNA laddering in CM (FIG. 4C). The anti-apoptotic effect of FADD-dn in CMs was MOI-dependent as demonstrated by DNA laddering (24-h hypoxia) (FIG. 4D). To confirm these results, we employed a specific caspase-8 inhibitor, zIETD-fmk. Both the activation of caspase-8 and CM apoptosis in response to 4 h hypoxia/SD were also completely inhibited by zIETD-fink (FIG. 4E). Thus, FADD signaling to caspase-8 is necessary for hypoxia/SD-induced CM apoptosis.

In contrast, the overexpression of FADD-wt induced significant DNA fragmentation even under normoxic conditions in the presence of serum (FIG. 4F). Hypoxia/SD did not further enhance DNA fragmentation induced by FADD-wt overexpression (FIG. 4F). These results establish that FADD is necessary for hypoxia/SD-induced activation of caspase-8 as well as CM apoptosis and is sufficient for CM apoptosis.

FADD-dn Inhibits Caspase-3 Activation Induced by Hypoxia and Serum-Deprivation

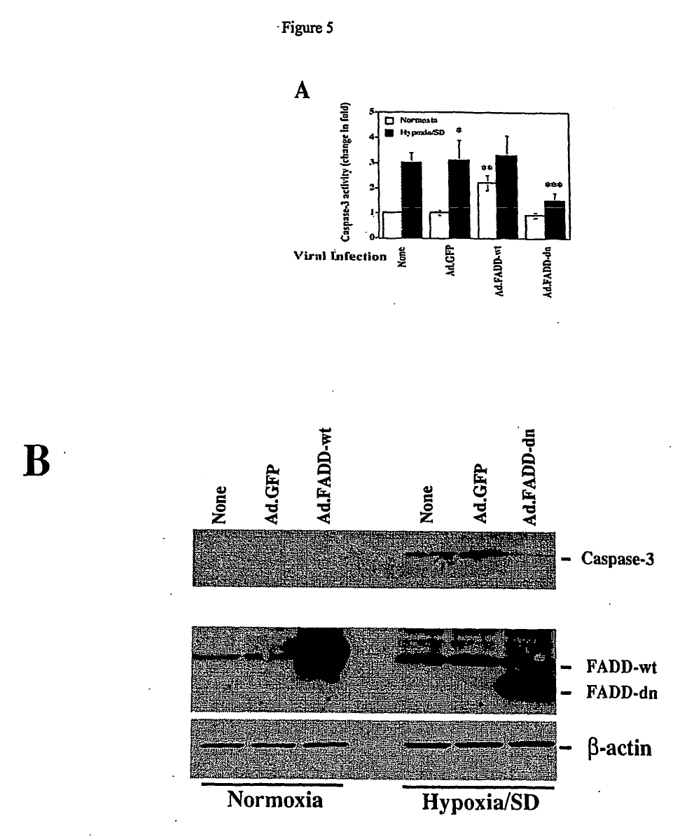

Given the degree of protection afforded by inhibition of FADD/caspase-8, we next examined whether these interventions were sufficient to inhibit caspase-3 activation. As shown in FIG. 5, in the presence of serum and normoxia, there was minimal activation of caspase-3 in uninfected control cells. Neither Ad.GFP nor Ad.FADD-dn infection significantly affected caspase-3 activity at baseline. However, hypoxia/SD induced significant activation of caspase-3 (up to 3-fold) in both uninfected and Ad.GFP-infected CM. Importantly, adenoviral expression of FADD-dn significantly inhibited the processing and activation of caspase-3 (FIG. 5). Moreover, consistent with its effect on DNA fragmentation, over-expression of FADD-wt resulted in a significant increase in caspase-3 activity although processing of procaspase-3 was not evident by immunoblotting likely reflecting the greater sensitivity of the enzymatic assay (FIG. 5).

FADD-dn and Caspase-8 Inhibitor Block Activation of Both Caspase-8 and -9

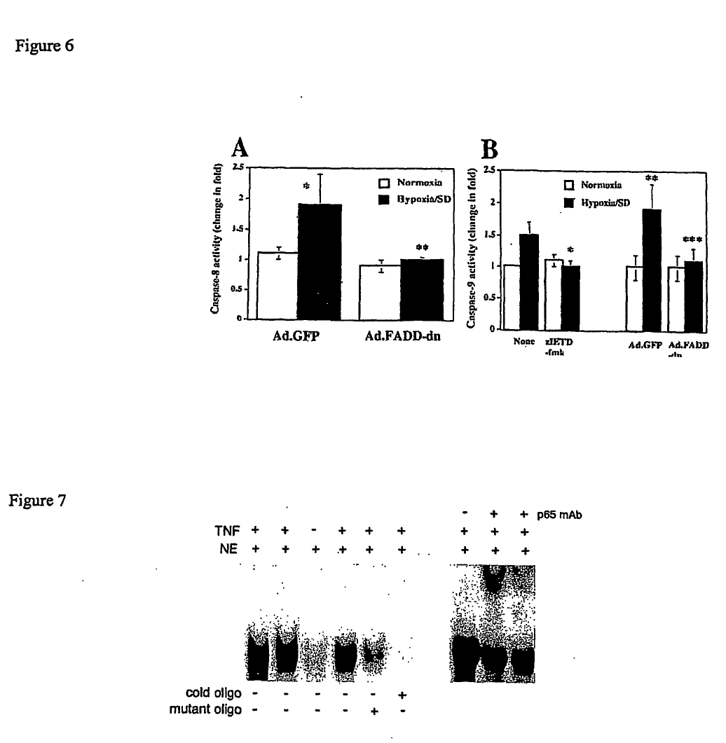

Although the intrinsic mitochondrial pathway appears to play a primary role in some models of hypoxia-induced apoptosis, this pathway did not induce more robust CM apoptosis even in the face of FADD inhibition. Therefore, we examined activation of caspase-9, the apical caspase in the mitochondrial pathway, in CM subjected to hypoxia/SD, as well as the effects of FADD-dn over-expression or zIETD-fink (a specific caspase-8 inhibitor) treatment. As expected, both zIETD-fink (FIG. 4E) and over-expression of FADD-dn (FIG. 6A) effectively blocked hypoxia/SD-induced caspase-8 activation in CM. Surprisingly, the activation of caspase-9 was also completely inhibited by pretreatment with zIETD-fmk or over-expression of FADD-dn (FIG. 6B). The observation that two completely different approaches (Ad.FADD-dn and zIETD-fink) directed at FADD/caspase-8 both inhibit caspase-9 demonstrates that both FADD and caspase-8 act upstream of caspase-9 and play a dominant role in CM apoptosis induced by hypoxia/SD.

Cell-Type Specific Inhibition of NF-κB Activation in Cardiomyocytes by Fas-Associated Death Domain Protein (FADD)

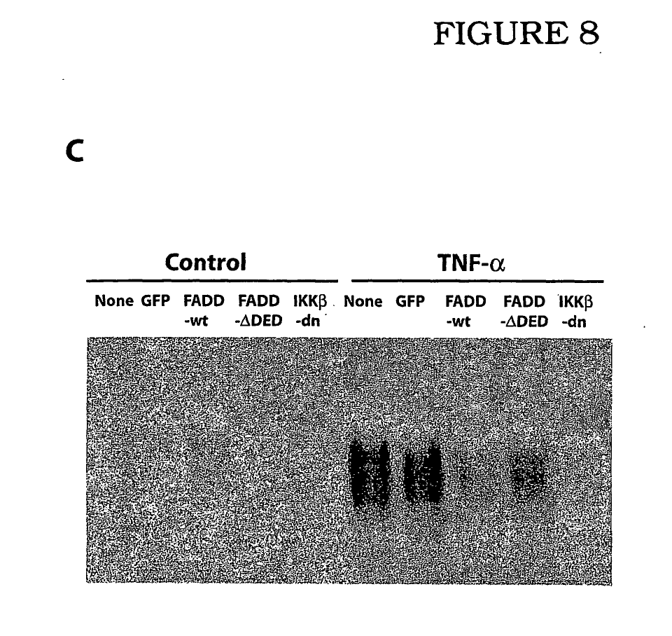

To examine the effect of FADD in cardiac inflammation, primary cardiomyocytes (CM) were infected with adenoviral vectors carrying either the wild-type FADD (Ad.FADD) or a truncation mutant lacking the DED (Ad.FADD-ΔDED) that is incapable of activating caspase-8 (see FIGS. 8A-8E). Both Ad.FADD and Ad.FADD-ΔDED effectively mediated expression of the appropriate size transgenes in rat neonatal CM in a MOI-dependent manner. Biological activity was confirmed by the ability of Ad.FADD but not Ad.FADD-ΔDED to activate caspase-8. To examine the role of FADD in inflammatory signaling, cultured neonatal rat CM were stimulated with TNF-α (50 ng/ml) for four hours. TNF-α stimulation activated NF-κB in CM as demonstrated by phosphorylation and degradation of IκB, nuclear translocation of the p65 NF-κB subunit, and increased mRNA for the NF-κB-dependent transcripts, VCAM-1 and ICAM-1, as assessed by quantitative real-time RT-PCR. Ad.FADD and Ad.FADD-ΔDED but not control Ad.GFP dramatically inhibited TNFα-induced p65 nuclear translocation and reduced ICAM-1 mRNA after stimulation. Ad.FADD also reduced VCAM-1 mRNA after stimulation. Although FADD-ΔDED exhibited a similar trend toward inhibition of VCAM-1, this did not achieve statistical significance (p=0.08). Together, these data suggest that FADD expression inhibits TNF-α induced activation of NF-κB in rat neonatal cardiomyocytes without affecting baseline NF-κB activity.

FADD's Effects on NF-κB are Cell-Type Specific

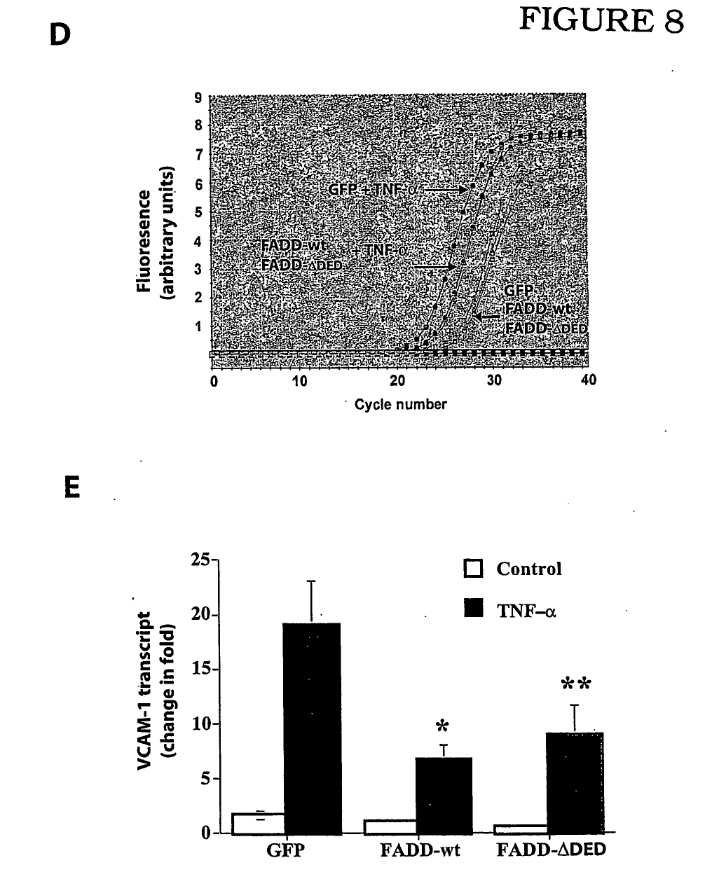

We next examined the effects of FADD expression on NF-κB activation in other cell types, including human umbilical vein endothelial cells (HUVEC), rat pulmonary artery smooth muscle cells (rPASMCs), and an immortalized human kidney tumor cell line (HEK293 cells). In HUVEC, FADD inhibited NF-κB activation in response to both TNF-α and LPS (FIG. 9A). Compared to wild-type FADD, FADD-ΔDED exhibited a more modest effect on TNF-α-induced NF-κB activation but a comparable inhibition in response to LPS. In contrast, FADD in smooth muscle cells did not inhibit TNF-α-mediated NF-κB activation although the expression of dominant negative IKKβ, did (FIG. 9B). Consistent with this finding, TNF-α induction of VCAM-1 mRNA was also unaffected in smooth muscle cells by expression of either FADD wild type or FADD-ΔDED. In both endothelial and smooth muscle cells, neither FADD constructs affected the basal level of NF-κB DNA binding activity in unstimulated cells. Interestingly, in human HEK293 cells, viral expression of FADD alone was sufficient to increase both nuclear translocation of p65 and NF-κB DNA binding activity in the absence of cytokine stimulation (FIGS. 9C and 9D). Thus, the regulatory effects of FADD on NF-κB activation appeared to be cell-type specific. FADD expression did not affect TNF-α-induced p65 nuclear translocation or VCAM-1/ICAM-1 mRNA levels in rat vascular smooth muscle cells or in human umbilical vein endothelial cells.

FADD Inhibits TNF-α-Induced Phosphorylation of IκB-

[symbol]

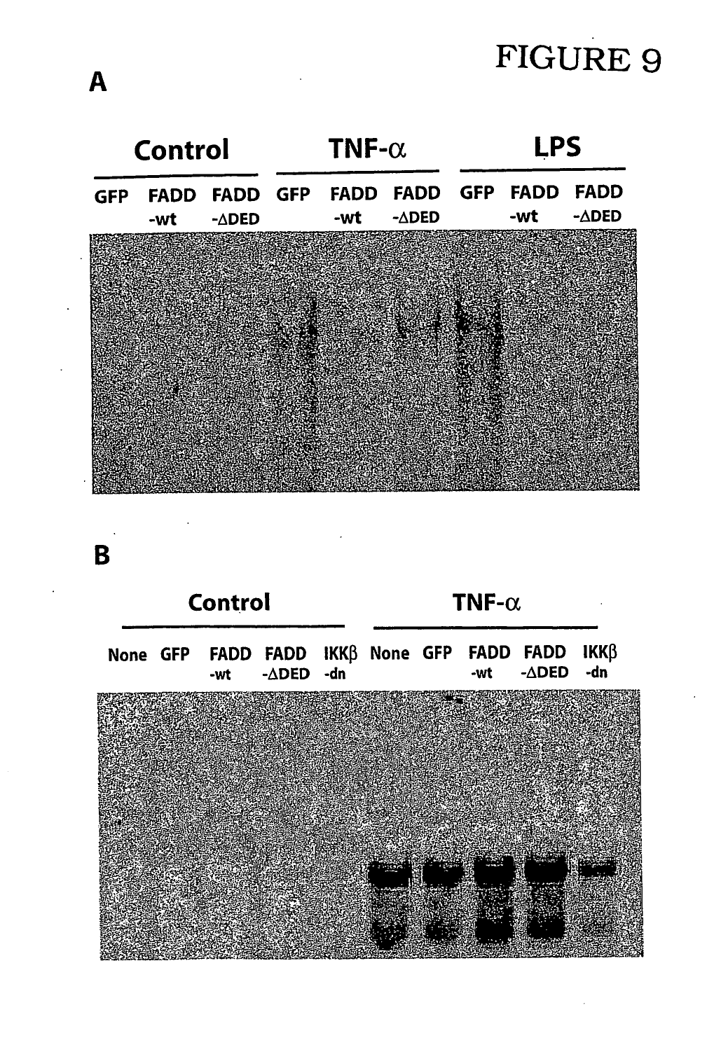

To explore the mechanisms by which FADD inhibits TNF-α-mediated NF-κB activation, we examined the effect of FADD on IκB- [symbol] phosphorylation as a critical step in NF-κB activation. As shown in FIG. 10, TNF- [symbol] resulted in an induction in the phosphorylation and in the slight degradation of IκB-α within five minutes. Adenoviral expression of FADD led to a dramatic decrease in the level of phospho-IκB- [symbol] but not total IκB- [symbol] as compared to TNF-α-stimulated GFP-expressing cells (FIG. 10).

FADD inhibits NIK- and IKKβ-Induced NF-κB Activation

To identify the level at which FADD inhibits NF-κB activation, we simultaneously infected CM with Ad.FADD and vectors encoding kinases in the NF-κB signaling cascade, NIK and IKKβ. Adenoviral expression of NIK or IKKβ alone was sufficient to induce significant NF-κB activation in CM as demonstrated by p65 nuclear translocation and increased VCAM-1/ICAM-1 mRNA levels. Simultaneously, over-expression of FADD had no effect on NIK or IKKβ-induced NFκB activation. Thus, FADD inhibits TNFα-induced NF-κB activation in CM but not vascular smooth muscle or endothelial cells through a mechanism independent of the DED and upstream of both NIK and IKKβ.

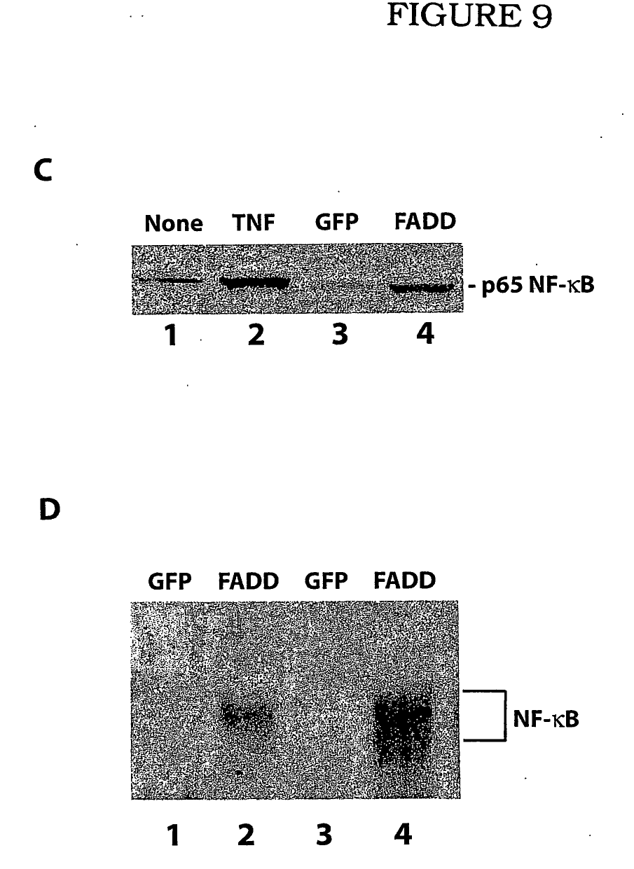

Furthermore, the expression of either NIK or IKKβ was sufficient to induce the phosphorylation of IκB- [symbol] (FIG. 11A). Co-expression of FADD inhibited IκB- [symbol] phosphorylation in response to both NIK and IKKβ, expression. Although the antibodies to total and phospho-IKKβ, did not recognize the endogenous rat IKKβ, they did detect the expressed human IKKβ, transgene and demonstrated that FADD inhibited IKKβ phosphorylation in response to IKKβ, expression (FIG. 11A). Furthermore, FADD expression significantly reduced NF-κB binding activity stimulated by expression of NIK and IKKβ (FIG. 11B). FADD-ΔDED also inhibited the stimulatory effect of the kinases but to a much lesser degree.

FADD-wt and FADD-dn Inhibit TNF-Induced NF-κB Activation in Cardiomyocytes

As noted above, chronic FADD has been reported to mediate both pro-inflammatory and anti-inflammatory effects in smooth muscle cells and endothelial cells, respectively (Schaub et al. (2000) Nature Med.; Bannerman et al. (2002) J. Clin. Invest). FADD blocked NF-κB activation in response to IL-1β but not TNF-α in endothelial cells. To examine the effect of FADD signaling on NF-κB and inflammatory mediators in primary CM, cultured neonatal rat CM were stimulated with TNF-α (50 ng/ml) for four hours. TNF-α stimulation activated NF-κB in CM as demonstrated by phosphorylation and degradation of IκB, nuclear translocation of the p65 NF-κB subunit, and increased mRNA for the NF-κB-dependent transcript, VCAM-1, as assessed by quantitative RT-PCR (QRT-PCR, data not shown). Expression of FADD dramatically inhibited TNF-α induced p65 nuclear translocation (FIG. 8A, upper panel). In FIG. 8A, the amount of nuclear proteins is controlled for by immunoblotting for Oct-1 (middle panel) and FADD expression is shown using the AU-epitope tag incorporated into the cDNA (lower panel). FADD expression also reduced VCAM-1 mRNA after stimulation by a mean of 80% (n=6, p<0.02), while transcription of a variety of control genes was unaffected. Similar results were obtained with expression of FADD-dn (which also inhibited p65 translocation and reduced VCAM-1 mRNA by 73%), suggesting this effect—in contrast to modulation of apoptosis—is not mediated via the DED.

Overall, the above experiments were designed to examine the effect of FADD, both wild type and a mutant lacking the death effector domain (ΔDED), on TNF-α-induced NFκB activation and NF-κB-dependent gene expression in primary rat cardiomyocytes. We found that over-expression of FADD significantly inhibited TNF-α-NF-κB signaling as demonstrated by decreased IκB- [symbol] phosphorylation, decreased NF-κB nuclear translocation and DNA-binding activity, and down-regulation of VCAM-1 mRNA level. FADD expression also inhibited NFκB activation initiated by two signaling molecules, IKK [symbol] and NIK. FADD exhibited a similar inhibitory effect on NF-κB activity in HUVEC but had no effect in rPASMCs and actually activated NF-κB in the human epithelial HEK293 cell line.

The over-expression of FADD mediates death receptor (DR)-induced activation of NF-κB in a variety of cell types. In FADD-deficient Jurkat cells (Wajant et al., supra) for example, DR-induced NF-κB activation is significantly reduced or abolished, suggesting an important role for FADD in mediating NF-κB activation in this setting. In 293 cells, FADD expression induces activation of NF-κB in a caspase-8-dependent manner (Hu et al., supra). In rat smooth muscle cells, stable expression of FADD induces production of the NF-κB-dependent chemokines, MCP-1 and IL-8, but only after 4 days of expression (Schaub et al., supra). Accordingly, our results showing that FADD inhibited NF-κB activation in response to TNF-α [symbol] in cardiomyocytes are surprising. FADD-ADED had a more modest effect on NF-κB activation, although it consistently inhibited NF-κB activation. This suggests that the DED domain of FADD is not essential for its inhibitory effect on NF-κB. Consistent with this, the caspase-8 inhibitor, IETD-fink, at a concentration (50 μM) sufficient to block caspase-8 activation and cardiomyocyte apoptosis (Chao et al., (2002) J Biol Chem 277:31639-31645), failed to reverse the FADD effect on NF-κB. The finding that FADD-ΔDED blocks both apoptosis and NF-κB activation in cardiomyocytes suggests that the FADD mutant as a useful therapeutic agent for the treatment of cardiac conditions characterized by co-existent cell death and inflammation, as often seen in tissue injury.

Our results further suggest that FADD may function by interfering with the assembly of the IKK signaling complex or through an intermediary. In this regard, we show that in HUVEC, FADD inhibited NF-κB activation downstream of either TNF- [symbol] or LPS (FIG. 9A). Although TNF- [symbol] and LPS signaling differ proximately, they both activate and converge at the level of IKK [symbol] (Takeda et al., (2003) Annu Rev Immunol 21:335-376; Karin et al., (2000) Annu Rev Immunol 18:621-663). Second, FADD expression inhibited IKK [symbol] phosphorylation in response to IKK [symbol] expression, thought to occur through auto-phosphorylation (Karin et al., supra). Both these lines of evidence suggest that FADD regulation of NF-κB activation may occur at the level of IKK [symbol] activation.

NF-κB Activation In Vivo

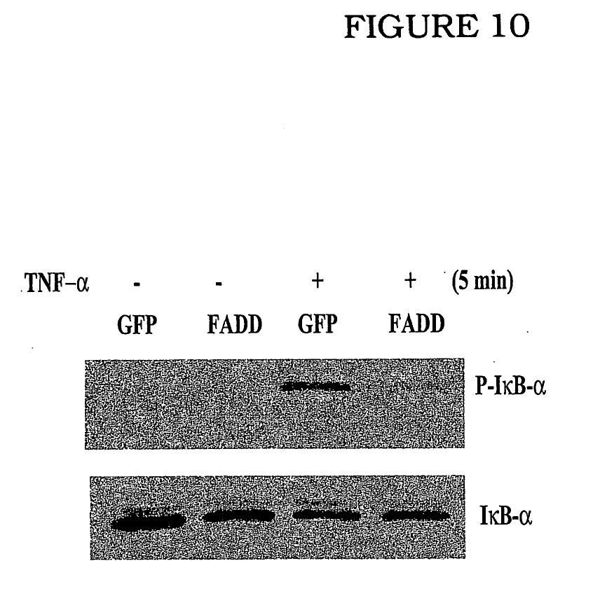

We have also established techniques for assessing the activation of NF-κB and downstream inflammatory effectors in the myocardium in vivo. For example, the presence of key cytoplasmic and nuclear NF-κB and IκB family members has been examined by immunoblotting and immunohistochemistry. As early as 30 minutes into reperfusion, significant degradation of cytosolic IκB- [symbol] is evident in myocardial tissue (FIG. 12A) and is followed by a subsequent increase in nuclear accumulation of the NF-κB p65 subunit (FIG. 12B). Both the decrease in cytosolic IκB-α and the increase in nuclear p65 are substantially blocked by injection of Ad.dnIKK-β prior to IRI (FIG. 12). NF-κB activation can also be localized to specific regions of myocardium and cells by confocal immunohistochemistry to identify subcellular localization of p65 (FIG. 12C). Confocal microscopy for GFP (left panel) which is co-expressed by Ad.dnIKK-β (and the other vectors described in this application) and for p65 (right panel, arrows) demonstrates that p65 remains predominantly in the cytoplasm of Ad.dnIKKβ-transduced (GFP-expressing) cells but moves to the nucleus in cells not expressing the transgene (seen in bottom half of right panel) after IRI (FIG. 12C). Using this approach, we correlated within specific cells the effects of transgene expression on NF-κB activation. Moreover, this approach can be combined with cell-specific markers to positively identify the cell-types involved (e.g., CM vs endothelial cells vs fibroblasts) and the presence of nuclear DNA fragmentation (e.g., TUNEL evidence of apoptosis).

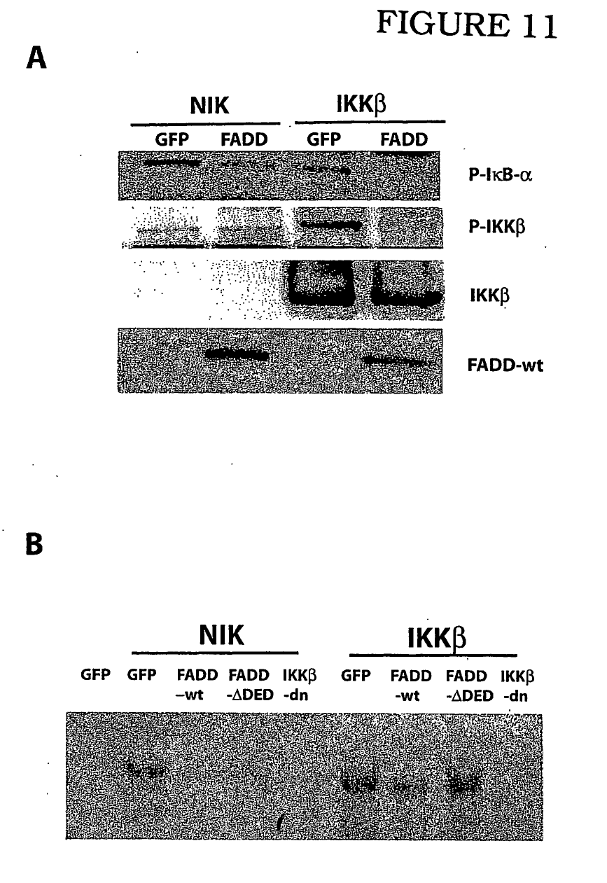

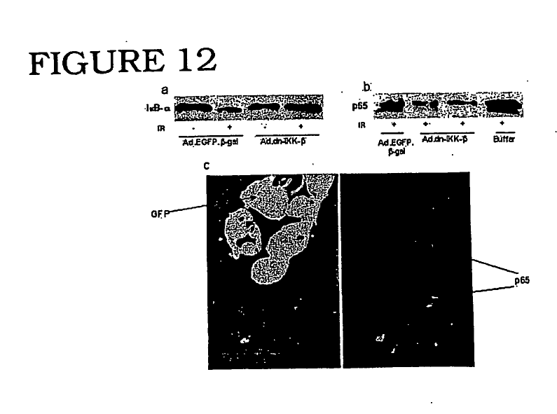

We have developed QRT-PCR primer/probe sets for a variety of NF-κB-dependent transcripts using rat sequences for molecules such as ICAM-1, VCAM-1, and MCP-1 (FIG. 13). This approach provides an extremely sensitive, specific, and accurate determination of mRNA levels for these transcripts. Increasing mRNA levels shift the amplification curves to the left, as seen after TNF stimulation of CM (FIG. 13). In this setting, we see a dramatic increase (left shift) in mRNA for ICAM-1 and VCAM-1 24 hr after ischemia induction (FIG. 14).

The observed changes in mRNA have corresponded to changes in immunohistochemical staining for adhesion molecules. We have also measured in vivo induction of the NF-κB-dependent inflammatory chemokine, MCP-1, since a quantitative ELISA assay specific for rat MCP-1 is commercially available. Tissue MCP-1 expression increased after IRI in ischemic myocardium in Ad.EGFP.β-gal treated rats compared with normal hearts (87.3±7.3 vs. 192.6±85.1 pg/ml, p<0.05, n-4 in each group). This increase was completely blocked in Ad.dnIKK-β treated animals (95.2±41.8 vs. 192.6±85.1 pg/ml, p<0.05), reducing chemoline concentration to levels seen in non-ischemic or normal myocardium. Finally, inflammatory infiltration by leukocytes (primarily neutrophils acutely) was assessed by immunohistochemistry and by measurement of myeloperoxidase activity (MPO), a specific marker for neutrophils. MPO activity was increased in ischemic regions following 30 minutes of ischemia and 24 hours of reperfusion in control virus treated rats compared with normal hearts. However, MPO activity in the ischemic area was decreased by 33% in Ad.dnIKK-β treated rats compared with rats injected with control virus (90.7±7.8 vs. 134.9±17.9 OD/minute/mg protein, p<0.05, n—4 in each group). Together these data show that NF-κB activation after IRI in vivo, as well as the induction of a variety of NF-κB-dependent pro-inflammatory mediators. Moreover, these studies also establish the feasibility of modulating NF-κB activation through gene transfer in the heart.

FADD-dn Reduces Infarction after IRI In Vivo at 24 hr

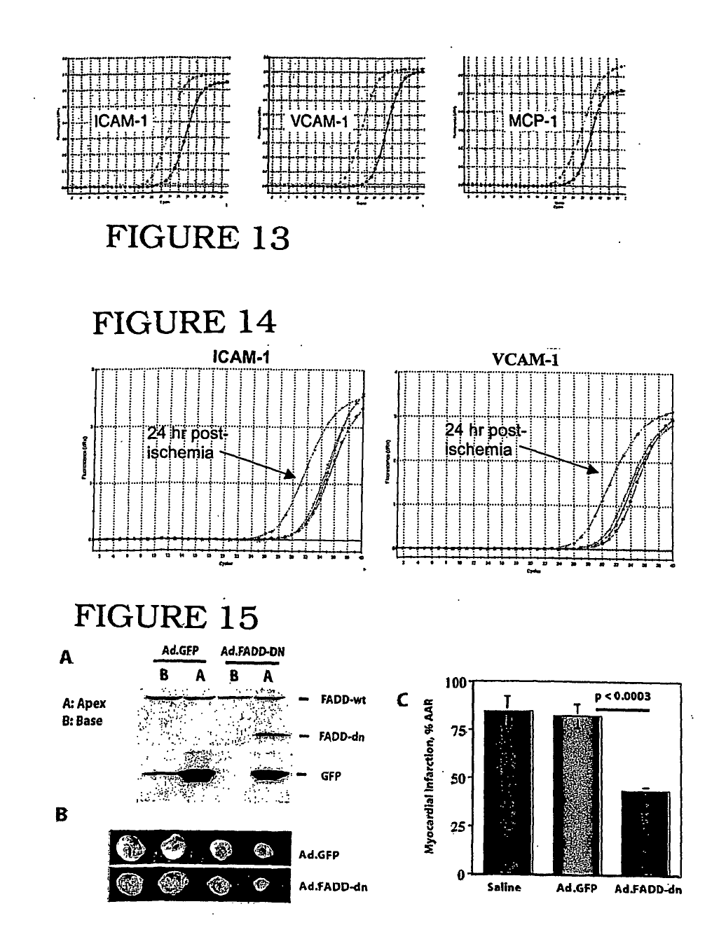

We have examined the effects of FADD-dn in vivo. Rats injected with Ad.FADD-dn, control virus, or saline, 48 hr prior to ischemia, were subjected to 30 min of ischemia and 24-hr reperfusion. Adenoviral gene transfer achieved significant expression of the FADD-dn construct and GFP, primarily in the apical region of injection (FIG. 15A). After adenoviral injection, FADD-dn expression in the apex was comparable to the expression level of the endogenous FADD (seen on the same anti-FADD blot migrating at a larger molecular weight, FIG. 15A, upper panel). LAD ligation produced a significant area of antero-apical ischemia as indicated by the absence of fluorescent microspheres. This area-at-risk (AAR) was not different among the three groups (data not shown). However, infarct size in hearts expressing FADD-dn was reduced by ˜50% (p<0.0003) (FIGS. 15B and 15C). Thus, FADD-dn expression in light of its potent effects on CM survival and its anti-inflammatory effects confers a substantial cardioprotective effect in vivo.

Screening and Identifying Compounds for the Treatment and Prevention of Cardiac Disorders

The present invention also provides methods for screening candidate compounds useful for reducing or preventing apoptosis of cardiomyocytes or for treating, reducing, or preventing cardiac inflammation, ischemic-reperfusion injury, heart failure, or a cardiac disorder in a mammal. Such compounds are typically identified by their ability to reduce the expression level of FADD (e.g., mRNA or protein) or the biological activity of FADD protein. Using such agents as lead compounds, the present screening methods also allow the identification of further novel, specific inhibitors of cardiac apoptosis and inflammation that function to treat, reduce, or prevent cardiac inflammation, ischemic-reperfusion injury, heart failure, or a cardiac disorder in a mammal. The method of screening may involve high-throughput techniques.

A number of methods are available for carrying out such screening assays. For example, these methods involve contacting one or more candidate compounds with a cardiomyocyte expressing an FADD gene (e.g., human) and measuring FADD gene expression or FADD protein activity using any technique known in the art. A compound that reduces FADD expression (e.g., mRNA or protein levels) or protein activity in a cardiomycyte that has been contacted relative to an untreated control is determined to be a candidate compound useful for reducing or preventing apoptosis of cardiomyocytes or for treating, reducing, or preventing cardiac inflammation, ischemic-reperfusion injury, heart failure, or a cardiac disorder in a mammal. Optionally, the cardiomyocyte may express an FADD fusion gene and the cardiomyocyte can be any mammalian cell, including for example, a rodent cell.

In one particular example, candidate compounds may be added at varying concentrations to the culture medium of cardiomyocytes expressing FADD. Gene expression of FADD is then measured, for example, by standard Northern blot analysis (Ausubel et al., supra), using any appropriate fragment prepared from the nucleic acid molecule of FADD as a hybridization probe or by real time PCR with appropriate primers. The level of gene expression in the presence of the candidate compound is compared to the level measured in a control culture medium lacking the candidate molecule. If desired, the effect of candidate compounds may, in the alternative, be measured at the level of FADD polypeptide or FADD activity using the same general approach and standard immunological techniques, such as Western blotting or immunoprecipitation with an antibody specific to FADD for example. For example, immunoassays may be used to detect or monitor the level of FADD. Polyclonal or monoclonal antibodies which are capable of binding to FADD may be used in any standard immunoassay format (e.g., ELISA or RIA assay) to measure the levels of FADD. FADD can also be measured using mass spectroscopy, high performance liquid chromatography, spectrophotometric or fluorometric techniques, or combinations thereof. Candidate compounds may also be identified by contacting a candidate compound with an FADD protein. Compounds that bind the FADD protein (e.g., human) are identified as candidate compounds for reducing or preventing apoptosis of cardiomyocytes or for treating, reducing, or preventing cardiac inflammation, ischemic-reperfusion injury, heart failure, or a cardiac disorder in a mammal.

In this particular regard, a candidate compound that binds to FADD may be identified using a chromatography-based technique. For example, a recombinant FADD may be purified by standard techniques from cells engineered to express FADD (e.g., those described above) and may be immobilized on a column. Alternatively, the naturally-occurring FADD may be immobilized on a column. A solution of candidate compounds is then passed through the column, and a compound specific for FADD is identified on the basis of its ability to bind to FADD and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography).

In a further example, a compound that interferes with FADD binding other signaling molecules involved in cardiac inflammation or cardiomyocyte apoptosis (e.g., to the DR, thereby reducing the biological activity of FADD), leading to a reduction in apoptosis in cardiomyocytes or to a reduction in cardiac inflammation, is useful according to the present invention. Given its ability to decrease the biological activity of FADD, such a molecule may be used, for example, as a therapeutic agent to treat, reduce, or prevent vascular disorders. As a specific example, a candidate compound may be contacted with two proteins, the first protein being a polypeptide substantially identical to FADD and the second protein being DR (i.e., a protein that binds the FADD under conditions that allow binding and that results in cardiomyocyte apoptosis). According to this particular screening method, the interaction between these two proteins is measured following the addition of a candidate compound. A decrease in the binding FADD to the second polypeptide following the addition of the candidate compound (relative to such binding in the absence of the compound) identifies the candidate compound as having the ability to inhibit the interaction between the two proteins, and thereby having the ability to reduce cardiomyocyte apoptosis. Preferably, the candidate compound also reduces cardiac inflammation. This screening assay may be carried out, for example, in a cell-free system or using a yeast two-hybrid system. If desired, one of the proteins or the candidate compound may be immobilized on a support as described above or may have a detectable group.

Alternatively, the screening methods of the invention may be used to identify candidate compounds that decrease the biological activity of FADD, for example, by reducing binding of FADD to the DR or by reducing FADD-mediated activation of caspase-8. Preferably, such reduction in biological activity is at least 10%, 20%, 30%, 40%, 50%, 60%, 70%, 80%, 90%, 95%, or 100% relative to an untreated control. For example, a candidate compound may be tested for its ability to decrease FADD activity in cells that naturally express FADD, after transfection with cDNA for FADD, or in cell-free solutions containing FADD, as described further below. The effect of a candidate compound on the binding or activation of FADD can be tested by radioactive and non-radiaoctive binding assays, competition assays, and receptor signaling assays.

As a specific example, mammalian cells (e.g., rodent cells) that express a nucleic acid encoding FADD are cultured in the presence of a candidate compound (e.g., a peptide, polypeptide, synthetic organic molecule, naturally occurring organic molecule, nucleic acid molecule, or component thereof). Cells may either endogenously express FADD or may alternatively be genetically engineered by any standard technique known in the art (e.g., transfection and viral infection) to overexpress FADD. The biological activity of FADD is measured in these cells and subsequently compared to such activity in control cells that have not been contacted by the candidate compound. A compound which promotes a decrease in the level of FADD activity as a result of reducing its synthesis or biological activity is considered useful in the invention.

Screening for new inhibitors and optimization of lead compounds may be assessed, for example, by assessing cardiac inflammation, cardiomyocyte apoptosis, or both using standard techniques. In addition, these candidate compounds may be tested for their ability to function as agents useful to treat, reduce, or prevent cardiac disorders (e.g., as described herein). Compounds which are identified as binding to FADD with an affinity constant less than or equal to 10 mM are considered particularly useful in the invention.

Potential therapeutic agents include organic molecules, peptides, peptide mimetics, polypeptides, and antibodies that bind to a nucleic acid sequence or polypeptide that encodes FADD and thereby inhibit or extinguish their activity. Potential agents also include small molecules that bind to and occupy the binding site of such polypeptides thereby preventing binding to cellular binding molecules, such that normal biological activity is prevented. Other potential analgesics include antisense molecules.

### Test Compounds and Extracts

In general, compounds capable of reducing hypertension are identified from large libraries of both natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Mernimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their analgesic activity should be employed whenever possible.

When a crude extract is found to have an analgesic activity, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract having therapeutic activity. Methods of fractionation and purification of such heterogenous extracts are known in the art. If desired, compounds shown to be useful agents for the treatment of pain are chemically modified according to methods known in the art.

### Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1-14. (canceled)

15. A method for treating, reducing, or preventing a cardiac disorder in a mammal, said method comprising administering to said mammal an effective amount of FADD or an anti-inflammatory FADD inhibitor.

16. The method of claim 15, wherein said cardiac disorder is a result of a chronic ischemia injury, an acute ischemia injury, an ischemia-reperfusion injury, a myocardial infarction, myocarditis, heart failure, cardiac transplantation, or an autoimmune disorder.

17-19. (canceled)

20. The method of claim 15, wherein said anti-inflammatory FADD inhibitor is a nucleic acid encoding a dominant negative FADD protein.

21. The method of claim 15, wherein said mammal is a human.

22-28. (canceled)

29. A human cardiomyocyte expressing a dominant negative FADD protein.

30. A human cardiomyocyte expressing a recombinant FADD protein.

31-41. (canceled)

42. A method for identifying a candidate compound for treating, reducing, or preventing cardiac inflammation in a mammal, said method comprising:
(a) contacting a cardiomyocyte expressing a FADD gene with a candidate compound; and
(b) measuring FADD gene expression or FADD protein activity in said a cardiomyocyte, a candidate compound that reduces said expression or said activity, relative to FADD expression or activity in a cardiomyocyte not contacted with said candidate compound, identifying said candidate compound as a candidate compound useful for treating, reducing, or preventing cardiac inflammation.

43-44. (canceled)

45. A method for identifying a candidate compound for treating, reducing, or preventing a cardiac disorder, said method comprising:
(a) contacting a cardiomyocyte expressing a FADD gene with a candidate compound; and
(b) measuring FADD gene expression or FADD protein activity in said a cardiomyocyte, a candidate compound that reduces said expression or said activity, relative to FADD expression or activity in a cardiomyocyte not contacted with said candidate compound, identifying said candidate compound as a candidate compound useful for treating, reducing, or preventing said cardiac disorder.

46. The method of any one of claims 42 or 45, wherein said FADD gene is a FADD fusion gene.

47. The method of any one of claims 42 or 45, wherein step (b) comprises measuring expression of FADD mRNA or protein.

48. The method of any one of claims 42 or 45, wherein said cardiomyocyte is a mammalian cell.

49. The method of claim 48, wherein said mammalian cell is a rodent cell.

50. (canceled)

51. A method for identifying a candidate compound for treating, reducing, or preventing cardiac inflammation, said method comprising:
(a) contacting FADD protein with a candidate compound; and
(b) determining whether said candidate compound binds said FADD protein, a candidate compound that binds said FADD protein being a candidate compound useful for treating, reducing, or preventing cardiac inflammation.

52-53. (canceled)

54. A method for identifying a candidate compound for treating, reducing, or preventing cardiac disorder, said method comprising:
(a) contacting FADD protein with a candidate compound; and
(b) determining whether said candidate compound binds said FADD protein, a candidate compound that binds said FADD protein being a candidate compound useful for treating, reducing, or preventing said cardiac disorder.

55. The method of claim 42, 45, 51, or 54, wherein said FADD is human FADD.

56-64. (canceled)
